SEQUENCE LISTING (278 sequences)

SEQ ID NO: 1 - DNA, 22 bp, Artificial Sequence
  Phosphorylated Oligonucleotide for Lung Cancer Panel
gggtgaggcagtctttactcac

SEQ ID NO: 2 - DNA, 24 bp, Artificial Sequence
  Phosphorylated Oligonucleotide for Lung Cancer Panel
gccgttgtacactcatcttcctag

SEQ ID NO: 3 - DNA, 23 bp, Artificial Sequence
  Phosphorylated Oligonucleotide for Lung Cancer Panel
ccaatgcagcgaacaatgttctg

SEQ ID NO: 4 - DNA, 27 bp, Artificial Sequence
  Phosphorylated Oligonucleotide for Lung Cancer Panel
tgcctttatacattgtagctgctgaaa

SEQ ID NO: 5 - DNA, 22 bp, Artificial Sequence
  Phosphorylated Oligonucleotide for Lung Cancer Panel
acaacaactgcagcaaagactg

SEQ ID NO: 6 - DNA, 22 bp, Artificial Sequence
  Phosphorylated Oligonucleotide for Lung Cancer Panel
gctctgcagcaaattcaaccac

SEQ ID NO: 7 - DNA, 22 bp, Artificial Sequence
  Phosphorylated Oligonucleotide for Lung Cancer Panel
gggtgtctctctgtggctttac

SEQ ID NO: 8 - DNA, 22 bp, Artificial Sequence
  Phosphorylated Oligonucleotide for Lung Cancer Panel
ctctgtaggctgcagttctcag

SEQ ID NO: 9 - DNA, 26 bp, Artificial Sequence
  Phosphorylated Oligonucleotide for Lung Cancer Panel
actccatcgagatttcactgtagcta

SEQ ID NO: 10 - DNA, 30 bp, Artificial Sequence
  Phosphorylated Oligonucleotide for Lung Cancer Panel
tctcttacctaaactcttcataatgcttgc

SEQ ID NO: 11 - DNA, 25 bp, Artificial Sequence
  Phosphorylated Oligonucleotide for Lung Cancer Panel
catacttaccatgccactttccctt

SEQ ID NO: 12 - DNA, 27 bp, Artificial Sequence
  Phosphorylated Oligonucleotide for Lung Cancer Panel
ctttttctgtttggcttgacttgactt

SEQ ID NO: 13 - DNA, 27 bp, Artificial Sequence
  Phosphorylated Oligonucleotide for Lung Cancer Panel
aatgactttctagtaactcagcagcat

SEQ ID NO: 14 - DNA, 29 bp, Artificial Sequence
  Phosphorylated Oligonucleotide for Lung Cancer Panel
cctcacagtaaaaataggtgattttggtc

SEQ ID NO: 15 - DNA, 28 bp, Artificial Sequence
  Phosphorylated Oligonucleotide for Lung Cancer Panel
cctattatgacttgtcacaatgtcacca

SEQ ID NO: 16 - DNA, 23 bp, Artificial Sequence
  Phosphorylated Oligonucleotide for Lung Cancer Panel
tagacgggactcgagtgatgatt

SEQ ID NO: 17 - DNA, 24 bp, Artificial Sequence
  Phosphorylated Oligonucleotide for Lung Cancer Panel
gggttactttgtgggagactttca

SEQ ID NO: 18 - DNA, 22 bp, Artificial Sequence
  Phosphorylated Oligonucleotide for Lung Cancer Panel
acaggtccacatccattcatcc

SEQ ID NO: 19 - DNA, 29 bp, Artificial Sequence
  Phosphorylated Oligonucleotide for Lung Cancer Panel
tagctgcagattatgaaatttaacagggt

SEQ ID NO: 20 - DNA, 25 bp, Artificial Sequence
  Phosphorylated Oligonucleotide for Lung Cancer Panel
gaatagggctgttcttgacaaaagg

SEQ ID NO: 21 - DNA, 22 bp, Artificial Sequence
  Phosphorylated Oligonucleotide for Lung Cancer Panel
ggtgacccttgtctctgtgttc

SEQ ID NO: 22 - DNA, 23 bp, Artificial Sequence
  Phosphorylated Oligonucleotide for Lung Cancer Panel
agggaccttaccttatacaccgt

SEQ ID NO: 23 - DNA, 22 bp, Artificial Sequence
  Phosphorylated Oligonucleotide for Lung Cancer Panel
ctggtaacatccacccagatca

SEQ ID NO: 24 - DNA, 27 bp, Artificial Sequence
  Phosphorylated Oligonucleotide for Lung Cancer Panel
ggagatgttgcttctcttaattccttg

SEQ ID NO: 25 - DNA, 19 bp, Artificial Sequence
  Phosphorylated Oligonucleotide for Lung Cancer Panel
tctggccaccatgcgaagc

SEQ ID NO: 26 - DNA, 19 bp, Artificial Sequence
  Phosphorylated Oligonucleotide for Lung Cancer Panel
ggcatgagctgcgtgatga

SEQ ID NO: 27 - DNA, 22 bp, Artificial Sequence
  Phosphorylated Oligonucleotide for Lung Cancer Panel
ggactatgtccgggaacacaaa

SEQ ID NO: 28 - DNA, 22 bp, Artificial Sequence
  Phosphorylated Oligonucleotide for Lung Cancer Panel
atggcaaactcttgctatccca

SEQ ID NO: 29 - DNA, 24 bp, Artificial Sequence
  Phosphorylated Oligonucleotide for Lung Cancer Panel
tgtcaagatcacagattttgggct

SEQ ID NO: 30 - DNA, 28 bp, Artificial Sequence
  Phosphorylated Oligonucleotide for Lung Cancer Panel
atgtgttaaacaatacagctagtgggaa

SEQ ID NO: 31 - DNA, 30 bp, Artificial Sequence
  Phosphorylated Oligonucleotide for Lung Cancer Panel
ggaaactgaattcaaaaagatcaaagtgct

SEQ ID NO: 32 - DNA, 26 bp, Artificial Sequence
  Phosphorylated Oligonucleotide for Lung Cancer Panel
ggaaatatacagcttgcaaggactct

SEQ ID NO: 33 - DNA, 27 bp, Artificial Sequence
  Phosphorylated Oligonucleotide for Lung Cancer Panel
tgagaaagttaaaattcccgtcgctat

SEQ ID NO: 34 - DNA, 22 bp, Artificial Sequence
  Phosphorylated Oligonucleotide for Lung Cancer Panel
ctgccagacatgagaaaaggtg

SEQ ID NO: 35 - DNA, 19 bp, Artificial Sequence
  Phosphorylated Oligonucleotide for Lung Cancer Panel
gaagcctacgtgatggcca

SEQ ID NO: 36 - DNA, 24 bp, Artificial Sequence
  Phosphorylated Oligonucleotide for Lung Cancer Panel
caggtactgggagccaatattgtc

SEQ ID NO: 37 - DNA, 22 bp, Artificial Sequence
  Phosphorylated Oligonucleotide for Lung Cancer Panel
cacagcagggtcttctctgttt

SEQ ID NO: 38 - DNA, 27 bp, Artificial Sequence
  Phosphorylated Oligonucleotide for Lung Cancer Panel
ccttctgcatggtattctttctcttcc

SEQ ID NO: 39 - DNA, 33 bp, Artificial Sequence
  Phosphorylated Oligonucleotide for Lung Cancer Panel
tgtcagcttattatattcaatttaaacccacct

SEQ ID NO: 40 - DNA, 22 bp, Artificial Sequence
  Phosphorylated Oligonucleotide for Lung Cancer Panel
caggtcaagaggagtacagtgc

SEQ ID NO: 41 - DNA, 23 bp, Artificial Sequence
  Phosphorylated Oligonucleotide for Lung Cancer Panel
caaagaatggtcctgcaccagta

SEQ ID NO: 42 - DNA, 27 bp, Artificial Sequence
  Phosphorylated Oligonucleotide for Lung Cancer Panel
aaggcctgctgaaaatgactgaatata

SEQ ID NO: 43 - DNA, 23 bp, Artificial Sequence
  Phosphorylated Oligonucleotide for Lung Cancer Panel
tcctcatgtactggtccctcatt

SEQ ID NO: 44 - DNA, 25 bp, Artificial Sequence
  Phosphorylated Oligonucleotide for Lung Cancer Panel
ggtgcactgtaataatccagactgt

SEQ ID NO: 45 - DNA, 23 bp, Artificial Sequence
  Phosphorylated Oligonucleotide for Lung Cancer Panel
gctgtatcgtcaaggcactcttg

SEQ ID NO: 46 - DNA, 30 bp, Artificial Sequence
  Phosphorylated Oligonucleotide for Lung Cancer Panel
aggtactggtggagtatttgatagtgtatt

SEQ ID NO: 47 - DNA, 22 bp, Artificial Sequence
  Phosphorylated Oligonucleotide for Lung Cancer Panel
ggtgcactgggactttggtaat

SEQ ID NO: 48 - DNA, 24 bp, Artificial Sequence
  Phosphorylated Oligonucleotide for Lung Cancer Panel
tccatctcttggaaactcccatct

SEQ ID NO: 49 - DNA, 24 bp, Artificial Sequence
  Phosphorylated Oligonucleotide for Lung Cancer Panel
tctgagaacaggaagttggtagct

SEQ ID NO: 50 - DNA, 27 bp, Artificial Sequence
  Phosphorylated Oligonucleotide for Lung Cancer Panel
cagcaagtttacaatgttcaaatgtgg

SEQ ID NO: 51 - DNA, 23 bp, Artificial Sequence
  Phosphorylated Oligonucleotide for Lung Cancer Panel
gggtgatgctattcagctacaga

SEQ ID NO: 52 - DNA, 27 bp, Artificial Sequence
  Phosphorylated Oligonucleotide for Lung Cancer Panel
tagttcgaatcatgcatgatgtctctg

SEQ ID NO: 53 - DNA, 22 bp, Artificial Sequence
  Phosphorylated Oligonucleotide for Lung Cancer Panel
gatgcagctgccttatgactca

SEQ ID NO: 54 - DNA, 25 bp, Artificial Sequence
  Phosphorylated Oligonucleotide for Lung Cancer Panel
caagctcagatctctattctgccaa

SEQ ID NO: 55 - DNA, 28 bp, Artificial Sequence
  Phosphorylated Oligonucleotide for Lung Cancer Panel
tgtctgaactgaagataatgactcacct

SEQ ID NO: 56 - DNA, 30 bp, Artificial Sequence
  Phosphorylated Oligonucleotide for Lung Cancer Panel
gatttaagcctgattgaacagttttcacaa

SEQ ID NO: 57 - DNA, 29 bp, Artificial Sequence
  Phosphorylated Oligonucleotide for Lung Cancer Panel
ggaaaaattgtgaagatctgtgactttgg

SEQ ID NO: 58 - DNA, 30 bp, Artificial Sequence
  Phosphorylated Oligonucleotide for Lung Cancer Panel
tctagaagcaacacctgactttagagatta

SEQ ID NO: 59 - DNA, 31 bp, Artificial Sequence
  Phosphorylated Oligonucleotide for Lung Cancer Panel
attttacagagtaacagactagctagagaca

SEQ ID NO: 60 - DNA, 31 bp, Artificial Sequence
  Phosphorylated Oligonucleotide for Lung Cancer Panel
agaaacagagaatctccattttagcacttac

SEQ ID NO: 61 - DNA, 27 bp, Artificial Sequence
  Phosphorylated Oligonucleotide for Lung Cancer Panel
acagcatgccaatctcttcataaatct

SEQ ID NO: 62 - DNA, 29 bp, Artificial Sequence
  Phosphorylated Oligonucleotide for Lung Cancer Panel
catgatgtgcatcattcatttgtttcatg

SEQ ID NO: 63 - DNA, 28 bp, Artificial Sequence
  Phosphorylated Oligonucleotide for Lung Cancer Panel
cctgaaggtattaacatcatttgctcca

SEQ ID NO: 64 - DNA, 24 bp, Artificial Sequence
  Phosphorylated Oligonucleotide for Lung Cancer Panel
ccagagccaagcatcattgagaaa

SEQ ID NO: 65 - DNA, 24 bp, Artificial Sequence
  Phosphorylated Oligonucleotide for Lung Cancer Panel
tgagcaagaggctttggagtattt

SEQ ID NO: 66 - DNA, 28 bp, Artificial Sequence
  Phosphorylated Oligonucleotide for Lung Cancer Panel
agagttattaacagtgcagtgtggaatc

SEQ ID NO: 67 - DNA, 26 bp, Artificial Sequence
  Phosphorylated Oligonucleotide Sequence for Colorectal Cancer Panel
actccatcgagatttcactgtagcta

SEQ ID NO: 68 - DNA, 30 bp, Artificial Sequence; identical to SEQ ID NO: 10
  Phosphorylated Oligonucleotide Sequence for Colorectal Cancer Panel

SEQ ID NO: 69 - DNA, 25 bp, Artificial Sequence
  Phosphorylated Oligonucleotide Sequence for Colorectal Cancer Panel
catacttaccatgccactttccctt

SEQ ID NO: 70 - DNA, 27 bp, Artificial Sequence
  Phosphorylated Oligonucleotide Sequence for Colorectal Cancer Panel
ctttttctgtttggcttgacttgactt

SEQ ID NO: 71 - DNA, 27 bp, Artificial Sequence
  Phosphorylated Oligonucleotide Sequence for Colorectal Cancer Panel
aatgactttctagtaactcagcagcat

SEQ ID NO: 72 - DNA, 29 bp, Artificial Sequence
  Phosphorylated Oligonucleotide Sequence for Colorectal Cancer Panel
cctcacagtaaaaataggtgattttggtc

SEQ ID NO: 73 - DNA, 28 bp, Artificial Sequence
  Phosphorylated Oligonucleotide Sequence for Colorectal Cancer Panel
cctattatgacttgtcacaatgtcacca

SEQ ID NO: 74 - DNA, 23 bp, Artificial Sequence
  Phosphorylated Oligonucleotide Sequence for Colorectal Cancer Panel
tagacgggactcgagtgatgatt

SEQ ID NO: 75 - DNA, 21 bp, Artificial Sequence
  Phosphorylated Oligonucleotide Sequence for Colorectal Cancer Panel
gtggtctcccataccctctca

SEQ ID NO: 76 - DNA, 23 bp, Artificial Sequence
  Phosphorylated Oligonucleotide Sequence for Colorectal Cancer Panel
acatggtctaagaggcagccata

SEQ ID NO: 77 - DNA, 20 bp, Artificial Sequence
  Phosphorylated Oligonucleotide Sequence for Colorectal Cancer Panel
gctggtgacacagcttatgc

SEQ ID NO: 78 - DNA, 20 bp, Artificial Sequence
  Phosphorylated Oligonucleotide Sequence for Colorectal Cancer Panel
ctccggagagacctgcaaag

SEQ ID NO: 79 - DNA, 22 bp, Artificial Sequence
  Phosphorylated Oligonucleotide Sequence for Colorectal Cancer Panel
tcctagagtaagccagggcttt

SEQ ID NO: 80 - DNA, 22 bp, Artificial Sequence
  Phosphorylated Oligonucleotide Sequence for Colorectal Cancer Panel
ccttacattcaaccgtgccatt

SEQ ID NO: 81 - DNA, 30 bp, Artificial Sequence
  Phosphorylated Oligonucleotide Sequence for Colorectal Cancer Panel
tctgacctacaaatatttacaggtaaccat

SEQ ID NO: 82 - DNA, 27 bp, Artificial Sequence
  Phosphorylated Oligonucleotide Sequence for Colorectal Cancer Panel
catttatctcctcaacaaccttccact

SEQ ID NO: 83 - DNA, 22 bp, Artificial Sequence
  Phosphorylated Oligonucleotide Sequence for Colorectal Cancer Panel
gccatgactgtcgctgtaaaga

SEQ ID NO: 84 - DNA, 26 bp, Artificial Sequence
  Phosphorylated Oligonucleotide Sequence for Colorectal Cancer Panel
ggtaactcaggactttgagttcagac

SEQ ID NO: 85 - DNA, 29 bp, Artificial Sequence
  Phosphorylated Oligonucleotide Sequence for Colorectal Cancer Panel
caccttctttctaaccttttcttatgtgc

SEQ ID NO: 86 - DNA, 27 bp, Artificial Sequence
  Phosphorylated Oligonucleotide Sequence for Colorectal Cancer Panel
cttataaagtgcagcttctgcatgatc

SEQ ID NO: 87 - DNA, 29 bp, Artificial Sequence
  Phosphorylated Oligonucleotide Sequence for Colorectal Cancer Panel
ggttttcttttctcctccaacctaatagt

SEQ ID NO: 88 - DNA, 23 bp, Artificial Sequence
  Phosphorylated Oligonucleotide Sequence for Colorectal Cancer Panel
gtcaagcagagaatgggtactca

SEQ ID NO: 89 - DNA, 30 bp, Artificial Sequence
  Phosphorylated Oligonucleotide Sequence for Colorectal Cancer Panel
agttctatagattctagtgcattcaagcac

SEQ ID NO: 90 - DNA, 27 bp, Artificial Sequence
  Phosphorylated Oligonucleotide Sequence for Colorectal Cancer Panel
gatatggtagacagagcctaaacatcc

SEQ ID NO: 91 - DNA, 26 bp, Artificial Sequence
  Phosphorylated Oligonucleotide Sequence for Colorectal Cancer Panel
cccacagaaacccatgtatgaagtac

SEQ ID NO: 92 - DNA, 23 bp, Artificial Sequence
  Phosphorylated Oligonucleotide Sequence for Colorectal Cancer Panel
cccaaaaaggtgacatggaaagc

SEQ ID NO: 93 - DNA, 23 bp, Artificial Sequence
  Phosphorylated Oligonucleotide Sequence for Colorectal Cancer Panel
ttgacagaacgggaagccctcat

SEQ ID NO: 94 - DNA, 30 bp, Artificial Sequence
  Phosphorylated Oligonucleotide Sequence for Colorectal Cancer Panel
gtcatgttttgataacctgacagacaataa

SEQ ID NO: 95 - DNA, 30 bp, Artificial Sequence
  Phosphorylated Oligonucleotide Sequence for Colorectal Cancer Panel
cgtgattcatttatttgttcaaagcaggaa

SEQ ID NO: 96 - DNA, 24 bp, Artificial Sequence
  Phosphorylated Oligonucleotide Sequence for Colorectal Cancer Panel
gccttgattgcaaacccttatgac

SEQ ID NO: 97 - DNA, 33 bp, Artificial Sequence; identical to SEQ ID NO: 39
  Phosphorylated Oligonucleotide Sequence for Colorectal Cancer Panel

SEQ ID NO: 98 - DNA, 22 bp, Artificial Sequence
  Phosphorylated Oligonucleotide Sequence for Colorectal Cancer Panel
caggtcaagaggagtacagtgc

SEQ ID NO: 99 - DNA, 23 bp, Artificial Sequence
  Phosphorylated Oligonucleotide Sequence for Colorectal Cancer Panel
caaagaatggtcctgcaccagta

SEQ ID NO: 100 - DNA, 27 bp, Artificial Sequence
  Phosphorylated Oligonucleotide Sequence for Colorectal Cancer Panel
aaggcctgctgaaaatgactgaatata

SEQ ID NO: 101 - DNA, 23 bp, Artificial Sequence
  Phosphorylated Oligonucleotide Sequence for Colorectal Cancer Panel
tcctcatgtactggtccctcatt

SEQ ID NO: 102 - DNA, 25 bp, Artificial Sequence
  Phosphorylated Oligonucleotide Sequence for Colorectal Cancer Panel
ggtgcactgtaataatccagactgt

SEQ ID NO: 103 - DNA, 23 bp, Artificial Sequence
  Phosphorylated Oligonucleotide Sequence for Colorectal Cancer Panel
gctgtatcgtcaaggcactcttg

SEQ ID NO: 104 - DNA, 30 bp, Artificial Sequence; identical to SEQ ID NO: 46
  Phosphorylated Oligonucleotide Sequence for Colorectal Cancer Panel

SEQ ID NO: 105 - DNA, 26 bp, Artificial Sequence
  Phosphorylated Oligonucleotide Sequence for Colorectal Cancer Panel
aacaacctaaaaccaactcttcccat

SEQ ID NO: 106 - DNA, 27 bp, Artificial Sequence
  Phosphorylated Oligonucleotide Sequence for Colorectal Cancer Panel
tgccatcaataatagcaagtcatttgc

SEQ ID NO: 107 - DNA, 25 bp, Artificial Sequence
  Phosphorylated Oligonucleotide Sequence for Colorectal Cancer Panel
tcttgtccagctgtatccagtatgt

SEQ ID NO: 108 - DNA, 29 bp, Artificial Sequence
  Phosphorylated Oligonucleotide Sequence for Colorectal Cancer Panel
gatgcttatttaaccttggcaatagcatt

SEQ ID NO: 109 - DNA, 22 bp, Artificial Sequence
  Phosphorylated Oligonucleotide Sequence for Colorectal Cancer Panel
ctccaaccaccaccagtttgta

SEQ ID NO: 110 - DNA, 24 bp, Artificial Sequence
  Phosphorylated Oligonucleotide Sequence for Colorectal Cancer Panel
ggaaggtcacactagggttttcat

SEQ ID NO: 111 - DNA, 29 bp, Artificial Sequence
  Phosphorylated Oligonucleotide Sequence for Colorectal Cancer Panel
gctcctagtacctgtagaggttaatatcc

SEQ ID NO: 112 - DNA, 28 bp, Artificial Sequence
  Phosphorylated Oligonucleotide Sequence for Colorectal Cancer Panel
gttatagatggtgaaacctgtttgttgg

SEQ ID NO: 113 - DNA, 22 bp, Artificial Sequence
  Phosphorylated Oligonucleotide Sequence for Colorectal Cancer Panel
cgacaagtgagagacaggatca

SEQ ID NO: 114 - DNA, 24 bp, Artificial Sequence
  Phosphorylated Oligonucleotide Sequence for Colorectal Cancer Panel
tcttgctggtgtgaaatgactgag

SEQ ID NO: 115 - DNA, 22 bp, Artificial Sequence
  Phosphorylated Oligonucleotide Sequence for Colorectal Cancer Panel
ggtgcactgggactttggtaat

SEQ ID NO: 116 - DNA, 24 bp, Artificial Sequence
  Phosphorylated Oligonucleotide Sequence for Colorectal Cancer Panel
tccatctcttggaaactcccatct

SEQ ID NO: 117 - DNA, 24 bp, Artificial Sequence
  Phosphorylated Oligonucleotide Sequence for Colorectal Cancer Panel
tctgagaacaggaagttggtagct

SEQ ID NO: 118 - DNA, 27 bp, Artificial Sequence
  Phosphorylated Oligonucleotide Sequence for Colorectal Cancer Panel
cagcaagtttacaatgttcaaatgtgg

SEQ ID NO: 119 - DNA, 23 bp, Artificial Sequence
  Phosphorylated Oligonucleotide Sequence for Colorectal Cancer Panel
gggtgatgctattcagctacaga

SEQ ID NO: 120 - DNA, 27 bp, Artificial Sequence
  Phosphorylated Oligonucleotide Sequence for Colorectal Cancer Panel
tagttcgaatcatgcatgatgtctctg

SEQ ID NO: 121 - DNA, 22 bp, Artificial Sequence
  Phosphorylated Oligonucleotide Sequence for Colorectal Cancer Panel
gatgcagctgccttatgactca

SEQ ID NO: 122 - DNA, 25 bp, Artificial Sequence
  Phosphorylated Oligonucleotide Sequence for Colorectal Cancer Panel
caagctcagatctctattctgccaa

SEQ ID NO: 123 - DNA, 28 bp, Artificial Sequence
  Phosphorylated Oligonucleotide Sequence for Colorectal Cancer Panel
tgtctgaactgaagataatgactcacct

SEQ ID NO: 124 - DNA, 30 bp, Artificial Sequence; identical to SEQ ID NO: 56
  Phosphorylated Oligonucleotide Sequence for Colorectal Cancer Panel

SEQ ID NO: 125 - DNA, 29 bp, Artificial Sequence
  Phosphorylated Oligonucleotide Sequence for Colorectal Cancer Panel
ggaaaaattgtgaagatctgtgactttgg

SEQ ID NO: 126 - DNA, 30 bp, Artificial Sequence; identical to SEQ ID NO: 58
  Phosphorylated Oligonucleotide Sequence for Colorectal Cancer Panel

SEQ ID NO: 127 - DNA, 31 bp, Artificial Sequence; identical to SEQ ID NO: 59
  Phosphorylated Oligonucleotide Sequence for Colorectal Cancer Panel

SEQ ID NO: 128 - DNA, 31 bp, Artificial Sequence; identical to SEQ ID NO: 60
  Phosphorylated Oligonucleotide Sequence for Colorectal Cancer Panel

SEQ ID NO: 129 - DNA, 27 bp, Artificial Sequence
  Phosphorylated Oligonucleotide Sequence for Colorectal Cancer Panel
acagcatgccaatctcttcataaatct

SEQ ID NO: 130 - DNA, 29 bp, Artificial Sequence
  Phosphorylated Oligonucleotide Sequence for Colorectal Cancer Panel
catgatgtgcatcattcatttgtttcatg

SEQ ID NO: 131 - DNA, 28 bp, Artificial Sequence
  Phosphorylated Oligonucleotide Sequence for Colorectal Cancer Panel
cctgaaggtattaacatcatttgctcca

SEQ ID NO: 132 - DNA, 24 bp, Artificial Sequence
  Phosphorylated Oligonucleotide Sequence for Colorectal Cancer Panel
ccagagccaagcatcattgagaaa

SEQ ID NO: 133 - DNA, 24 bp, Artificial Sequence
  Phosphorylated Oligonucleotide Sequence for Colorectal Cancer Panel
tgagcaagaggctttggagtattt

SEQ ID NO: 134 - DNA, 28 bp, Artificial Sequence
  Phosphorylated Oligonucleotide Sequence for Colorectal Cancer Panel
agagttattaacagtgcagtgtggaatc

SEQ ID NO: 135 - DNA, 22 bp, Artificial Sequence
  Phosphorylated Oligonucleotide Sequence for Pan-Cancer Panel
agtatgcgctgaagctccattt

SEQ ID NO: 136 - DNA, 26 bp, Artificial Sequence
  Phosphorylated Oligonucleotide Sequence for Pan-Cancer Panel
caggttagggtgtttgatctctttca

SEQ ID NO: 137 - DNA, 22 bp, Artificial Sequence
  Phosphorylated Oligonucleotide Sequence for Pan-Cancer Panel
cgtgttgaagtcctcgttgtct

SEQ ID NO: 138 - DNA, 23 bp, Artificial Sequence
  Phosphorylated Oligonucleotide Sequence for Pan-Cancer Panel
gagatctgagtggccatgtacag

SEQ ID NO: 139 - DNA, 22 bp, Artificial Sequence
  Phosphorylated Oligonucleotide Sequence for Pan-Cancer Panel
gatctcgtcagccatggagtac

SEQ ID NO: 140 - DNA, 22 bp, Artificial Sequence
  Phosphorylated Oligonucleotide Sequence for Pan-Cancer Panel
ccagcactgaggttagaagctg

SEQ ID NO: 141 - DNA, 24 bp, Artificial Sequence
  Phosphorylated Oligonucleotide Sequence for Pan-Cancer Panel
agttcttgaaagaagctgcagtca

SEQ ID NO: 142 - DNA, 24 bp, Artificial Sequence
  Phosphorylated Oligonucleotide Sequence for Pan-Cancer Panel
tattccaacgaggttttgtgcagt

SEQ ID NO: 143 - DNA, 26 bp, Artificial Sequence
  Phosphorylated Oligonucleotide Sequence for Pan-Cancer Panel
cccgttctatatcatcactgagttca

SEQ ID NO: 144 - DNA, 26 bp, Artificial Sequence
  Phosphorylated Oligonucleotide Sequence for Pan-Cancer Panel
cctgtggatgaagtttttcttctcca

SEQ ID NO: 145 - DNA, 24 bp, Artificial Sequence
  Phosphorylated Oligonucleotide Sequence for Pan-Cancer Panel
cagaagattcgcagaagctcatct

SEQ ID NO: 146 - DNA, 27 bp, Artificial Sequence
  Phosphorylated Oligonucleotide Sequence for Pan-Cancer Panel
aatcagaggcctgaaaccaatctaaat

SEQ ID NO: 147 - DNA, 22 bp, Artificial Sequence
  Phosphorylated Oligonucleotide Sequence for Pan-Cancer Panel
gggtgaggcagtctttactcac

SEQ ID NO: 148 - DNA, 24 bp, Artificial Sequence
  Phosphorylated Oligonucleotide Sequence for Pan-Cancer Panel
gccgttgtacactcatcttcctag

SEQ ID NO: 149 - DNA, 22 bp, Artificial Sequence
  Phosphorylated Oligonucleotide Sequence for Pan-Cancer Panel
gggtgtctctctgtggctttac

SEQ ID NO: 150 - DNA, 22 bp, Artificial Sequence
  Phosphorylated Oligonucleotide Sequence for Pan-Cancer Panel
ctctgtaggctgcagttctcag

SEQ ID NO: 151 - DNA, 22 bp, Artificial Sequence
  Phosphorylated Oligonucleotide Sequence for Pan-Cancer Panel
ttggcacaacaactgcagcaaa

SEQ ID NO: 152 - DNA, 25 bp, Artificial Sequence
  Phosphorylated Oligonucleotide Sequence for Pan-Cancer Panel
agcaaattcaaccaccagaacattg

SEQ ID NO: 153 - DNA, 27 bp, Artificial Sequence
  Phosphorylated Oligonucleotide Sequence for Pan-Cancer Panel
aatgactttctagtaactcagcagcat

SEQ ID NO: 154 - DNA, 29 bp, Artificial Sequence
  Phosphorylated Oligonucleotide Sequence for Pan-Cancer Panel
cctcacagtaaaaataggtgattttggtc

SEQ ID NO: 155 - DNA, 28 bp, Artificial Sequence
  Phosphorylated Oligonucleotide Sequence for Pan-Cancer Panel
cctattatgacttgtcacaatgtcacca

SEQ ID NO: 156 - DNA, 23 bp, Artificial Sequence
  Phosphorylated Oligonucleotide Sequence for Pan-Cancer Panel
tagacgggactcgagtgatgatt

SEQ ID NO: 157 - DNA, 26 bp, Artificial Sequence
  Phosphorylated Oligonucleotide Sequence for Pan-Cancer Panel
actccatcgagatttcactgtagcta

SEQ ID NO: 158 - DNA, 30 bp, Artificial Sequence; identical to SEQ ID NO: 10
  Phosphorylated Oligonucleotide Sequence for Pan-Cancer Panel

SEQ ID NO: 159 - DNA, 25 bp, Artificial Sequence
  Phosphorylated Oligonucleotide Sequence for Pan-Cancer Panel
catacttaccatgccactttccctt

SEQ ID NO: 160 - DNA, 27 bp, Artificial Sequence
  Phosphorylated Oligonucleotide Sequence for Pan-Cancer Panel
ctttttctgtttggcttgacttgactt

SEQ ID NO: 161 - DNA, 26 bp, Artificial Sequence
  Phosphorylated Oligonucleotide Sequence for Pan-Cancer Panel
gaaatttaacagggtgttgttgtgca

SEQ ID NO: 162 - DNA, 24 bp, Artificial Sequence
  Phosphorylated Oligonucleotide Sequence for Pan-Cancer Panel
ctgttcatctgacagctgggaata

SEQ ID NO: 163 - DNA, 28 bp, Artificial Sequence
  Phosphorylated Oligonucleotide Sequence for Pan-Cancer Panel
tgtttgtttgtttaactttgtgtcgcta

SEQ ID NO: 164 - DNA, 26 bp, Artificial Sequence
  Phosphorylated Oligonucleotide Sequence for Pan-Cancer Panel
cactatactgacgtctccaacatgag

SEQ ID NO: 165 - DNA, 23 bp, Artificial Sequence
  Phosphorylated Oligonucleotide Sequence for Pan-Cancer Panel
ccaggacgtttgtggaaaacaag

SEQ ID NO: 166 - DNA, 29 bp, Artificial Sequence
  Phosphorylated Oligonucleotide Sequence for Pan-Cancer Panel
atgaatgagaaagaggacatcttatggtg

SEQ ID NO: 167 - DNA, 20 bp, Artificial Sequence
  Phosphorylated Oligonucleotide Sequence for Pan-Cancer Panel
cccagcagaggttctagacg

SEQ ID NO: 168 - DNA, 24 bp, Artificial Sequence
  Phosphorylated Oligonucleotide Sequence for Pan-Cancer Panel
gctgttatccaggtttctgttgtt

SEQ ID NO: 169 - DNA, 21 bp, Artificial Sequence
  Phosphorylated Oligonucleotide Sequence for Pan-Cancer Panel
caggagctttcaccaacctgt

SEQ ID NO: 170 - DNA, 21 bp, Artificial Sequence
  Phosphorylated Oligonucleotide Sequence for Pan-Cancer Panel
cgctgtttcatgctcctcctt

SEQ ID NO: 171 - DNA, 24 bp, Artificial Sequence
  Phosphorylated Oligonucleotide Sequence for Pan-Cancer Panel
gagatgtccctcttgtcactaacg

SEQ ID NO: 172 - DNA, 22 bp, Artificial Sequence
  Phosphorylated Oligonucleotide Sequence for Pan-Cancer Panel
ccagctgatggctttctcttcc

SEQ ID NO: 173 - DNA, 21 bp, Artificial Sequence
  Phosphorylated Oligonucleotide Sequence for Pan-Cancer Panel
cccaatcaccagatcgaatgg

SEQ ID NO: 174 - DNA, 25 bp, Artificial Sequence
  Phosphorylated Oligonucleotide Sequence for Pan-Cancer Panel
cctctctttcgtgtcaaaggacttc

SEQ ID NO: 175 - DNA, 21 bp, Artificial Sequence
  Phosphorylated Oligonucleotide Sequence for Pan-Cancer Panel
cccacagcatggacatacatg

SEQ ID NO: 176 - DNA, 21 bp, Artificial Sequence
  Phosphorylated Oligonucleotide Sequence for Pan-Cancer Panel
gaaggacttgggcattcaggt

SEQ ID NO: 177 - DNA, 25 bp, Artificial Sequence
  Phosphorylated Oligonucleotide Sequence for Pan-Cancer Panel
ctaaccatcatttcgttttgccaga

SEQ ID NO: 178 - DNA, 22 bp, Artificial Sequence
  Phosphorylated Oligonucleotide Sequence for Pan-Cancer Panel
tccaaaggtttacccacctgtc

SEQ ID NO: 179 - DNA, 21 bp, Artificial Sequence
  Phosphorylated Oligonucleotide Sequence for Pan-Cancer Panel
ctcagccaagggagctcgaga

SEQ ID NO: 180 - DNA, 20 bp, Artificial Sequence
  Phosphorylated Oligonucleotide Sequence for Pan-Cancer Panel
ctggaactgctacatgtgcg

SEQ ID NO: 181 - DNA, 19 bp, Artificial Sequence
  Phosphorylated Oligonucleotide Sequence for Pan-Cancer Panel
gatgactggcacgctccat

SEQ ID NO: 182 - DNA, 21 bp, Artificial Sequence
  Phosphorylated Oligonucleotide Sequence for Pan-Cancer Panel
gctgtgtggttagacggcttc

SEQ ID NO: 183 - DNA, 23 bp, Artificial Sequence
  Phosphorylated Oligonucleotide Sequence for Pan-Cancer Panel
ccgggtacctttccatttcagtg

SEQ ID NO: 184 - DNA, 30 bp, Artificial Sequence
  Phosphorylated Oligonucleotide Sequence for Pan-Cancer Panel
gcttattcctcttttctcctcttcatctag

SEQ ID NO: 185 - DNA, 27 bp, Artificial Sequence
  Phosphorylated Oligonucleotide Sequence for Pan-Cancer Panel
ggaaaaggaaataaggaacatggcaga

SEQ ID NO: 186 - DNA, 20 bp, Artificial Sequence
  Phosphorylated Oligonucleotide Sequence for Pan-Cancer Panel
gggtaaccttcccggtatga

SEQ ID NO: 187 - DNA, 22 bp, Artificial Sequence
  Phosphorylated Oligonucleotide Sequence for Pan-Cancer Panel
cagctccacaatgcagatgaga

SEQ ID NO: 188 - DNA, 24 bp, Artificial Sequence
  Phosphorylated Oligonucleotide Sequence for Pan-Cancer Panel
cttctggctctttgagaatgtggt

SEQ ID NO: 189 - DNA, 20 bp, Artificial Sequence
  Phosphorylated Oligonucleotide Sequence for Pan-Cancer Panel
gaggaagcctatgtgcggaa

SEQ ID NO: 190 - DNA, 22 bp, Artificial Sequence
  Phosphorylated Oligonucleotide Sequence for Pan-Cancer Panel
cgttgcctttatcctcccagat

SEQ ID NO: 191 - DNA, 30 bp, Artificial Sequence
  Phosphorylated Oligonucleotide Sequence for Pan-Cancer Panel
ggacaaatggaagataaggagaaaaagagg

SEQ ID NO: 192 - DNA, 21 bp, Artificial Sequence
  Phosphorylated Oligonucleotide Sequence for Pan-Cancer Panel
gtccgcagcgtcacacagaag

SEQ ID NO: 193 - DNA, 19 bp, Artificial Sequence
  Phosphorylated Oligonucleotide Sequence for Pan-Cancer Panel
ccgaggcaatgtagcggtc

SEQ ID NO: 194 - DNA, 19 bp, Artificial Sequence
  Phosphorylated Oligonucleotide Sequence for Pan-Cancer Panel
cttgggcctacagctgacc

SEQ ID NO: 195 - DNA, 21 bp, Artificial Sequence
  Phosphorylated Oligonucleotide Sequence for Pan-Cancer Panel
gatgggcttcctcttctcagc

SEQ ID NO: 196 - DNA, 20 bp, Artificial Sequence
  Phosphorylated Oligonucleotide Sequence for Pan-Cancer Panel
agggtgtgtgggtctaggag

SEQ ID NO: 197 - DNA, 22 bp, Artificial Sequence
  Phosphorylated Oligonucleotide Sequence for Pan-Cancer Panel
ccagcactcacaaattcctggt

SEQ ID NO: 198 - DNA, 22 bp, Artificial Sequence
  Phosphorylated Oligonucleotide Sequence for Pan-Cancer Panel
ccaggcagccattaaggaagac

SEQ ID NO: 199 - DNA, 22 bp, Artificial Sequence
  Phosphorylated Oligonucleotide Sequence for Pan-Cancer Panel
ggtgacccttgtctctgtgttc

SEQ ID NO: 200 - DNA, 23 bp, Artificial Sequence
  Phosphorylated Oligonucleotide Sequence for Pan-Cancer Panel
agggaccttaccttatacaccgt

SEQ ID NO: 201 - DNA, 22 bp, Artificial Sequence
  Phosphorylated Oligonucleotide Sequence for Pan-Cancer Panel
ctggtaacatccacccagatca

SEQ ID NO: 202 - DNA, 27 bp, Artificial Sequence
  Phosphorylated Oligonucleotide Sequence for Pan-Cancer Panel
ggagatgttgcttctcttaattccttg

SEQ ID NO: 203 - DNA, 19 bp, Artificial Sequence
  Phosphorylated Oligonucleotide Sequence for Pan-Cancer Panel
catgcgaagccacactgac

SEQ ID NO: 204 - DNA, 20 bp, Artificial Sequence
  Phosphorylated Oligonucleotide Sequence for Pan-Cancer Panel
gttcccggacatagtccagg

SEQ ID NO: 205 - DNA, 30 bp, Artificial Sequence; identical to SEQ ID NO: 31
  Phosphorylated Oligonucleotide Sequence for Pan-Cancer Panel

SEQ ID NO: 206 - DNA, 26 bp, Artificial Sequence
  Phosphorylated Oligonucleotide Sequence for Pan-Cancer Panel
ggaaatatacagcttgcaaggactct

SEQ ID NO: 207 - DNA, 27 bp, Artificial Sequence
  Phosphorylated Oligonucleotide Sequence for Pan-Cancer Panel
tgagaaagttaaaattcccgtcgctat

SEQ ID NO: 208 - DNA, 22 bp, Artificial Sequence
  Phosphorylated Oligonucleotide Sequence for Pan-Cancer Panel
ctgccagacatgagaaaaggtg

SEQ ID NO: 209 - DNA, 24 bp, Artificial Sequence
  Phosphorylated Oligonucleotide Sequence for Pan-Cancer Panel
tgtttcagggcatgaactacttgg

SEQ ID NO: 210 - DNA, 23 bp, Artificial Sequence
  Phosphorylated Oligonucleotide Sequence for Pan-Cancer Panel
acctccttactttgcctccttct

SEQ ID NO: 211 - DNA, 21 bp, Artificial Sequence
  Phosphorylated Oligonucleotide Sequence for Pan-Cancer Panel
gtggtctcccataccctctca

SEQ ID NO: 212 - DNA, 21 bp, Artificial Sequence
  Phosphorylated Oligonucleotide Sequence for Pan-Cancer Panel
agccatagggcataagctgtg

SEQ ID NO: 213 - DNA, 29 bp, Artificial Sequence
  Phosphorylated Oligonucleotide Sequence for Pan-Cancer Panel
atgttaccataaatcaaaaatgcaccaca

SEQ ID NO: 214 - DNA, 26 bp, Artificial Sequence
  Phosphorylated Oligonucleotide Sequence for Pan-Cancer Panel
actttggattggctcgagatatcatg

SEQ ID NO: 215 - DNA, 23 bp, Artificial Sequence
  Phosphorylated Oligonucleotide Sequence for Pan-Cancer Panel
atctttgttgctgtccttccact

SEQ ID NO: 216 - DNA, 29 bp, Artificial Sequence
  Phosphorylated Oligonucleotide Sequence for Pan-Cancer Panel
atctttaaaatgcacgtactcaccatttg

SEQ ID NO: 217 - DNA, 30 bp, Artificial Sequence
  Phosphorylated Oligonucleotide Sequence for Pan-Cancer Panel
ttggaaactcccatttgagatcatattcat

SEQ ID NO: 218 - DNA, 28 bp, Artificial Sequence
  Phosphorylated Oligonucleotide Sequence for Pan-Cancer Panel
gcctattcctaactgactcatcatttca

SEQ ID NO: 219 - DNA, 25 bp, Artificial Sequence
  Phosphorylated Oligonucleotide Sequence for Pan-Cancer Panel
ccctgacaacatagttggaatcact

SEQ ID NO: 220 - DNA, 29 bp, Artificial Sequence
  Phosphorylated Oligonucleotide Sequence for Pan-Cancer Panel
cacagtaaataacactctggtgtcattct

SEQ ID NO: 221 - DNA, 23 bp, Artificial Sequence
  Phosphorylated Oligonucleotide Sequence for Pan-Cancer Panel
agacaaatggtgagtacgtgcat

SEQ ID NO: 222 - DNA, 30 bp, Artificial Sequence
  Phosphorylated Oligonucleotide Sequence for Pan-Cancer Panel
tcctcagataatgagtacttctacgttgat

SEQ ID NO: 223 - DNA, 30 bp, Artificial Sequence
  Phosphorylated Oligonucleotide Sequence for Pan-Cancer Panel
gagttctatagattctagtgcattcaagca

SEQ ID NO: 224 - DNA, 27 bp, Artificial Sequence
  Phosphorylated Oligonucleotide Sequence for Pan-Cancer Panel
gatatggtagacagagcctaaacatcc

SEQ ID NO: 225 - DNA, 26 bp, Artificial Sequence
  Phosphorylated Oligonucleotide Sequence for Pan-Cancer Panel
cccacagaaacccatgtatgaagtac

SEQ ID NO: 226 - DNA, 23 bp, Artificial Sequence
  Phosphorylated Oligonucleotide Sequence for Pan-Cancer Panel
cccaaaaaggtgacatggaaagc

SEQ ID NO: 227 - DNA, 23 bp, Artificial Sequence
  Phosphorylated Oligonucleotide Sequence for Pan-Cancer Panel
ttgacagaacgggaagccctcat

SEQ ID NO: 228 - DNA, 30 bp, Artificial Sequence; identical to SEQ ID NO: 94
  Phosphorylated Oligonucleotide Sequence for Pan-Cancer Panel

SEQ ID NO: 229 - DNA, 30 bp, Artificial Sequence; identical to SEQ ID NO: 95
  Phosphorylated Oligonucleotide Sequence for Pan-Cancer Panel

SEQ ID NO: 230 - DNA, 24 bp, Artificial Sequence
  Phosphorylated Oligonucleotide Sequence for Pan-Cancer Panel
gccttgattgcaaacccttatgac

SEQ ID NO: 231 - DNA, 30 bp, Artificial Sequence; identical to SEQ ID NO: 81
  Phosphorylated Oligonucleotide Sequence for Pan-Cancer Panel

SEQ ID NO: 232 - DNA, 27 bp, Artificial Sequence
  Phosphorylated Oligonucleotide Sequence for Pan-Cancer Panel
catttatctcctcaacaaccttccact

SEQ ID NO: 233 - DNA, 22 bp, Artificial Sequence
  Phosphorylated Oligonucleotide Sequence for Pan-Cancer Panel
gccatgactgtcgctgtaaaga

SEQ ID NO: 234 - DNA, 26 bp, Artificial Sequence
  Phosphorylated Oligonucleotide Sequence for Pan-Cancer Panel
ggtaactcaggactttgagttcagac

SEQ ID NO: 235 - DNA, 29 bp, Artificial Sequence
  Phosphorylated Oligonucleotide Sequence for Pan-Cancer Panel
caccttctttctaaccttttcttatgtgc

SEQ ID NO: 236 - DNA, 27 bp, Artificial Sequence
  Phosphorylated Oligonucleotide Sequence for Pan-Cancer Panel
cttataaagtgcagcttctgcatgatc

SEQ ID NO: 237 - DNA, 29 bp, Artificial Sequence
  Phosphorylated Oligonucleotide Sequence for Pan-Cancer Panel
ggttttcttttctcctccaacctaatagt

SEQ ID NO: 238 - DNA, 23 bp, Artificial Sequence
  Phosphorylated Oligonucleotide Sequence for Pan-Cancer Panel
gtcaagcagagaatgggtactca

SEQ ID NO: 239 - DNA, 22 bp, Artificial Sequence
  Phosphorylated Oligonucleotide Sequence for Pan-Cancer Panel
tcctcatgtactggtccctcat

SEQ ID NO: 240 - DNA, 25 bp, Artificial Sequence
  Phosphorylated Oligonucleotide Sequence for Pan-Cancer Panel
ggtgcactgtaataatccagactgt

SEQ ID NO: 241 - DNA, 23 bp, Artificial Sequence
  Phosphorylated Oligonucleotide Sequence for Pan-Cancer Panel
gctgtatcgtcaaggcactcttg

SEQ ID NO: 242 - DNA, 30 bp, Artificial Sequence; identical to SEQ ID NO: 46
  Phosphorylated Oligonucleotide Sequence for Pan-Cancer Panel

SEQ ID NO: 243 - DNA, 23 bp, Artificial Sequence
  Phosphorylated Oligonucleotide Sequence for Pan-Cancer Panel
caaagaatggtcctgcaccagta

SEQ ID NO: 244 - DNA, 27 bp, Artificial Sequence
  Phosphorylated Oligonucleotide Sequence for Pan-Cancer Panel
aaggcctgctgaaaatgactgaatata

SEQ ID NO: 245 - DNA, 30 bp, Artificial Sequence
  Phosphorylated Oligonucleotide Sequence for Pan-Cancer Panel
gaattttctaaaggtatctctctcggtgta

SEQ ID NO: 246 - DNA, 24 bp, Artificial Sequence
  Phosphorylated Oligonucleotide Sequence for Pan-Cancer Panel
ccagttacctcttggtcagtcatc

SEQ ID NO: 247 - DNA, 27 bp, Artificial Sequence
  Phosphorylated Oligonucleotide Sequence for Pan-Cancer Panel
cttaatagggtggttctcttcccaaag

SEQ ID NO: 248 - DNA, 30 bp, Artificial Sequence
  Phosphorylated Oligonucleotide Sequence for Pan-Cancer Panel
acacttaaaaagggtaaaggcagaatcata

SEQ ID NO: 249 - DNA, 28 bp, Artificial Sequence
  Phosphorylated Oligonucleotide Sequence for Pan-Cancer Panel
atccgcaaatgacttgctattattgatg

SEQ ID NO: 250 - DNA, 26 bp, Artificial Sequence
  Phosphorylated Oligonucleotide Sequence for Pan-Cancer Panel
cccaggattcttacagaaaacaagtg

SEQ ID NO: 251 - DNA, 22 bp, Artificial Sequence
  Phosphorylated Oligonucleotide Sequence for Pan-Cancer Panel
cctcacctctatggtgggatca

SEQ ID NO: 252 - DNA, 24 bp, Artificial Sequence
  Phosphorylated Oligonucleotide Sequence for Pan-Cancer Panel
cgccaattaaccctgattactggt

SEQ ID NO: 253 - DNA, 22 bp, Artificial Sequence
  Phosphorylated Oligonucleotide Sequence for Pan-Cancer Panel
ggtgcactgggactttggtaat

SEQ ID NO: 254 - DNA, 24 bp, Artificial Sequence
  Phosphorylated Oligonucleotide Sequence for Pan-Cancer Panel
tccatctcttggaaactcccatct

SEQ ID NO: 255 - DNA, 24 bp, Artificial Sequence
  Phosphorylated Oligonucleotide Sequence for Pan-Cancer Panel
tctgagaacaggaagttggtagct

SEQ ID NO: 256 - DNA, 27 bp, Artificial Sequence
  Phosphorylated Oligonucleotide Sequence for Pan-Cancer Panel
cagcaagtttacaatgttcaaatgtgg

SEQ ID NO: 257 - DNA, 23 bp, Artificial Sequence
  Phosphorylated Oligonucleotide Sequence for Pan-Cancer Panel
gggtgatgctattcagctacaga

SEQ ID NO: 258 - DNA, 27 bp, Artificial Sequence
  Phosphorylated Oligonucleotide Sequence for Pan-Cancer Panel
tagttcgaatcatgcatgatgtctctg

SEQ ID NO: 259 - DNA, 22 bp, Artificial Sequence
  Phosphorylated Oligonucleotide Sequence for Pan-Cancer Panel
gatgcagctgccttatgactca

SEQ ID NO: 260 - DNA, 25 bp, Artificial Sequence
  Phosphorylated Oligonucleotide Sequence for Pan-Cancer Panel
caagctcagatctctattctgccaa

SEQ ID NO: 261 - DNA, 28 bp, Artificial Sequence
  Phosphorylated Oligonucleotide Sequence for Pan-Cancer Panel
tgtctgaactgaagataatgactcacct

SEQ ID NO: 262 - DNA, 30 bp, Artificial Sequence; identical to SEQ ID NO: 56
  Phosphorylated Oligonucleotide Sequence for Pan-Cancer Panel

SEQ ID NO: 263 - DNA, 29 bp, Artificial Sequence
  Phosphorylated Oligonucleotide Sequence for Pan-Cancer Panel
ggaaaaattgtgaagatctgtgactttgg

SEQ ID NO: 264 - DNA, 30 bp, Artificial Sequence; identical to SEQ ID NO: 58
  Phosphorylated Oligonucleotide Sequence for Pan-Cancer Panel

SEQ ID NO: 265 - DNA, 27 bp, Artificial Sequence
  Phosphorylated Oligonucleotide Sequence for Pan-Cancer Panel
taagggaaaatgacaaagaacagctca

SEQ ID NO: 266 - DNA, 29 bp, Artificial Sequence
  Phosphorylated Oligonucleotide Sequence for Pan-Cancer Panel
gctgagatcagccaaattcagttattttt

SEQ ID NO: 267 - DNA, 30 bp, Artificial Sequence
  Phosphorylated Oligonucleotide Sequence for Pan-Cancer Panel
cttttgatgacattgcatacattcgaaaga

SEQ ID NO: 268 - DNA, 28 bp, Artificial Sequence
  Phosphorylated Oligonucleotide Sequence for Pan-Cancer Panel
cagttatcttttcagttcaatgcatgct

SEQ ID NO: 269 - DNA, 20 bp, Artificial Sequence
  Phosphorylated Oligonucleotide Sequence for Pan-Cancer Panel
tacgcagcctgtacccagtg

SEQ ID NO: 270 - DNA, 20 bp, Artificial Sequence
  Phosphorylated Oligonucleotide Sequence for Pan-Cancer Panel
ttgtggtagcagtggatgca

SEQ ID NO: 271 - DNA, 24 bp, Artificial Sequence
  Phosphorylated Oligonucleotide Sequence for Pan-Cancer Panel
ccctccttcctagagagttagagt

SEQ ID NO: 272 - DNA, 23 bp, Artificial Sequence
  Phosphorylated Oligonucleotide Sequence for Pan-Cancer Panel
caagagagcaacacccacactta

SEQ ID NO: 273 - DNA, 21 bp, Artificial Sequence
  Phosphorylated Oligonucleotide Sequence for Pan-Cancer Panel
cccagctgggtgaactttgag

SEQ ID NO: 274 - DNA, 24 bp, Artificial Sequence
  Phosphorylated Oligonucleotide Sequence for Pan-Cancer Panel
cagctgaaggtaatgagcacaaag

SEQ ID NO: 275 - DNA, 22 bp, Artificial Sequence
  Phosphorylated Oligonucleotide Sequence for Pan-Cancer Panel
catttttggcttcctggccttt

SEQ ID NO: 276 - DNA, 21 bp, Artificial Sequence
  Phosphorylated Oligonucleotide Sequence for Pan-Cancer Panel
ggtgggtgtctttatggcctt

SEQ ID NO: 277 - DNA, 22 bp, Artificial Sequence
  Phosphorylated Oligonucleotide Sequence for Pan-Cancer Panel
ccagtcccttacttgttcagct

SEQ ID NO: 278 - DNA, 22 bp, Artificial Sequence
  Phosphorylated Oligonucleotide Sequence for Pan-Cancer Panel
tgccaaagacagcccatcattt

(19) United States
(12) Patent Application Publication
(10) Pub. No.: US 2016/0098516 A1
(43) Pub. Date: Apr. 7, 2016

(54) METHODS AND SYSTEMS FOR DETECTION OF A GENETIC MUTATION

(71) Applicant: THE REGENTS OF THE UNIVERSITY OF CALIFORNIA, Oakland, CA (US)

(72) Inventors: Il-Jin KIM, Pacifica, CA (US); David JABLONS, San Francisco, CA (US); Pedro Juan Mendez ROMERO, San Francisco, CA (US); Jun-Hee YOON, San Francisco, CA (US)

(21) Appl. No.: 14/867,934

(22) Filed: Sep. 28, 2015

Related U.S. Application Data
(60) Provisional application No. 62/056,314, filed on Sep. 26, 2014.

(51) Int. Cl. G06F 19/22; C12N 15/10; C12Q 1/68

(52) U.S. Cl. CPC G06F 19/22; C12Q 1/6874; C12N 15/1003; C12Q 1/6806; C12N 15/1065

(57) ABSTRACT

Methods and systems for the detection of genetic mutations from a tissue sample (e.g., a preserved tissue sample) are provided. The method includes the steps of a) extracting a nucleic acid from a tissue or biological sample; b) preparing a targeted nucleic acid amplicon library from the extracted nucleic acid; c) sequencing the targeted nucleic acid amplicon library to produce tissue sample target nucleic acid sequence data; and d) analyzing the sample target nucleic acid sequence data to determine whether it contains a mutation (e.g., a mutation associated with a risk for a particular disease). The methods described herein advantageously can be performed in less than 36 hours.

### CROSS-REFERENCES TO RELATED APPLICATIONS

This application claims the benefit under 35 U.S.C. §119(e) of U.S. Provisional Application No. 62/056,314 filed on Sep. 26, 2014, which is incorporated herein by reference in its entirety for all purposes.

### FIELD OF THE INVENTION

Provided herein are methods and systems of genetic analysis. More specifically, provided herein are methods and systems for the detection of genetic mutations using tissue samples.

### BACKGROUND

Recently, treatment strategies of human disease are quickly moving into personalized medicine, such as targeted therapy in human cancers. Gefitinib and Erlotinib, for example, are well-used receptor tyrosine kinase (RTK) inhibitors that target EGFR mutations in lung cancer patients. Also, lung cancer patients with EML4-ALK fusion are known to be responsive to Crizotinib, a MET-ALK inhibitor. Many anti-cancer drugs in the market or under development are target-specific drugs. Thus, it is very important to expedite the genetic analysis of clinical specimens by using faster and more robust techniques.

Formalin-fixed, paraffin-embedded (FFPE) tissues are the most frequently used sample types in clinical genetic analysis. It is known that genomic DNA from FFPE tissues is highly degraded and of low quality. This limits the application of genomic DNA extracted from FFPE tissues in clinical genetic analysis. Moreover, extracting DNA from FFPE specimens via commercially available methods is an expensive and time-consuming process. Often, these processes involve toxic chemicals such as phenol or chloroform, which delay robust processing of patient samples. Therefore, there is a need for the development of a fast, easy, robust, and cost-effective method to prepare genomic DNA from FFPE samples for genetic analysis.

The emergence of next-generation sequencing (NGS) has changed the paradigm for genetic and genomic studies in many medical and life science fields. NGS has revolutionized and maximized the sequencing applications of human, animal, microbiological and agrogenomic samples. While previous genetic technologies such as Sanger sequencing mainly cover small regions on a single gene, the NGS can cover the whole exome (all exons of the genome) and even the whole genome. Genome-wide coverage of NGS applications enables for broadening of the scope of genetic and genomic studies of diseases. As many human diseases such as cancers are mainly caused by accumulation of genetic alterations in the key driver or main pathway regulators, it is highly anticipated that new therapeutic targets and diagnostic markers will be discovered using NGS. There have been many NGS projects identifying previously unreported genetic alterations (e.g., mutations, polymorphisms, amplification, chromosomal rearrangement, and gene fusions) that could be used for either therapeutic targets or diagnostic markers for human diseases such as cancer.

While the whole genome or exome sequencing is still widely used for many studies, the trend of NGS is now quickly moving toward targeted sequencing. Targeted sequencing, focusing on small but important gene sets or genetic regions, is a very powerful approach to screen disease-related key genes. Most of the NGS applications for the patient (e.g., cancer patients) screening are now being done by targeted NGS rather than an exome or whole genome sequencing. A fast reduction of cost and experimental time and an availability of the targeted sequencing are fueling the use of NGS for many genetic applications.

Although NGS is promising and is becoming more popular in many life science applications, several factors such as complicated sample preparation, high cost, and time-consuming data analyses, prevent its application from being used more routinely in clinical and research settings. Therefore, it is crucial that the current methods are improved or new methods for faster, more robust and accurate NGS applications are developed.

Moreover, NGS data analysis also presents a hurdle in using NGS. Thus, there is a need for the development of new, easy, and robust NGS data analysis tools that makes the NGS application more general and essential in many biological and clinical fields. Although targeted sequencing is becoming more dominant and popular in genetic screening in human diseases, data analysis has been mainly executed by programs or algorithms developed for the whole exome or genome sequencing.

Thus, a development of a robust targeted sequencing analysis tool will be very important for many applications of targeted sequencing, such as, for example, cancer diagnostics, personalized medicine, and prenatal screening.

### SUMMARY OF THE INVENTION

Provided herein are methods and systems for determining the presence of a mutation ((e.g., a mutation associated with the risk for a disease) in a a target nucleic acid from a tissue sample (e.g., a preserved tissue sample). In a first aspect, provided herein is a method for extracting nucleic acid from a preserved tissue sample. The method includes the steps of a) incubating the preserved tissue sample with a tissue digestion solution to form a tissue digestion mixture; b) heating the tissue digestion mixture at 80 to 110° C. for 1-30 minutes; c) adding a protease solution comprising a proteinase to the tissue digestion mixture to form a protein degradation mixture; d) incubating the protein degradation mixture at 50 to 70° C. for 1-30 minutes; and e) incubating the protein degradation mixture at 80 to 110° C. for 1-30 minutes; thereby extracting nucleic acid from the preserved tissue sample.

In some embodiments, the tissue digestion solution is selected from i) a tissue digestion solution comprising NaCl at a concentration of 10 mM to 140 mM, Na₂HPO₄ at a concentration of 0.5 mM to 10 mM, KH₂PO₄ at a concentration of 0.1 mM to 5 mM, and Tween 20; ii) a tissue digestion solution comprising NaCl at a concentration of 10 mM to 140 mM, Na₂HPO₄ at a concentration of 0.5 mM to 10 mM, KH₂PO₄ at a concentration of 0.1 mM to 5 mM, and Triton-X100; iii) a tissue digestion solution comprising NaCl at a concentration of 10 mM to 140 mM, Na₂HPO₄ at a concentration of 0.5 mM to 10 mM, and KH₂PO₄ at a concentration of 0.1 mM to 5 mM; iv) a tissue digestion solution comprising TAPS sodium salt at a concentration of 0.5 mM to 25 mM, DTT at a concentration of 0.05 mM to 5 mM, and KCl at a concentration of 0.2 mM to 200 mM; v) a tissue digestion solution comprising HEPES buffer at a concentration of 1 mM to 100 mM; vi) a tissue digestion solution comprising HEPES buffer at a concentration of 1 mM to 100 mM and Triton-X100; vii) a tissue digestion solution comprising HEPES buffer at a concentration of 1 mM to 100 mM and Tween 20; viii) a tissue digestion solution comprising TAPS sodium salt at a concentration of 0.5 mM to 25 mM, DTT at a concentration of 0.05 mM to 5 mM, KCl at a concentration of 0.2 mM to 200 mM, and Triton-X100; ix) a tissue digestion solution comprising a TAPS sodium salt at a concentration of 0.5 mM to 25 mM, DTT at a concentration of 0.05 mM to 5 mM, KCl at a concentration of 0.2 mM to 200 mM, and Tween 20; and x) a tissue digestion solution comprising a TAPS sodium salt at a concentration of 0.5 mM to 25 mM, KCl at a concentration of 0.2 mM to 200 mM, β-Mercaptoethanol at a concentration of 0.1 mM to 1 mM and Triton X-100.

In certain embodiments, the protease solution is selected from the group consisting of: a) a protease solution including Proteinase K at a concentration of 5 mg/ml to 60 mg/ml, Tris-HCl at a concentration of 1 mM to 50 mM and EDTA at a concentration of 0.1 to 10 mM; b) a protease solution including Proteinase K at a concentration of 5 mg/ml to 60 mg/ml; c) a protease solution including Proteinase K at a concentration of 5 mg/ml to 60 mg/ml and Tris-HCl at a concentration of 1 mM to 50 mM; d) a protease solution including Proteinase K at a concentration of 5 mg/ml to 60 mg/ml and EDTA at a concentration of 0.1 mM to 10 mM; e) a protease solution including Proteinase K at a concentration of 5 mg/ml to 60 mg/ml, Tris-HCl at a concentration of 0.2 mM to 50 mM, CaCl2 at a concentration of 0.1 mM to 10 mM and glycerol at a concentration of 20% to 70%.

In some embodiments, the heating (b) is at 99° C. for 5 to 30 minutes. In certain embodiments, the incubating the protein degradation mixture (c) is at 60° C. for 5 to 30 minutes. In some embodiments, the incubating the protein degradation mixture (d) is at 99° C. for 5 to 30 minutes.

In another aspect, provided herein is a method for making a targeted nucleic acid amplicon library from a tissue sample, the method includes the steps of: a) amplifying nucleic acid extracted from a tissue sample, the step of amplification using 5′ phosphorylated oligonucleotides that target a nucleic acid of interest; and b) directly ligating an oligonucleotide comprising an adaptor nucleic acid and a bar code nucleic acid to each of the amplified target nucleic acids, thereby making a targeted nucleic acid amplicon library. In certain embodiments, the method further includes the step of purifying the amplified target nucleic acid of (a) prior to directly ligating an oligonucleotide (b).

In another aspect, provided herein is a method of detecting a mutation in a tissue sample target nucleic acid sequence without preprocessing of sequence data, the method including the steps of: (a) obtaining a tissue sample target nucleic acid sequence data and database target nucleic acid sequence data, where the database target nucleic acid sequence data is located in a mutation database; (b) comparing the tissue sample target nucleic acid sequence data with the database target nucleic acid sequence data to determine if the sample target nucleic acid sequence data contains a registered mutation from the mutation database; (c) determining the reliability of the mutation that is registered in the mutation database by determining the mutant allele frequency of the mutation that is registered in the mutation database; and (d) generating a result as to whether the tissue sample target nucleic acid sequence data contains a mutation, thereby detecting the mutation.

In another aspect, provided herein is a computing system that includes one or more processors; memory; and one more programs. The one or more programs of the computing system are stored in the memory and are configured to be executed by the one or more processors for detecting a mutation in a tissue sample target nucleic acid sequence. The one or more programs include instructions for detecting a mutation in a tissue sample target nucleic acid sequence including: (a) obtaining a tissue sample target nucleic acid sequence data and database target nucleic acid sequence data, where the database target nucleic acid sequence data is located in a mutation database; (b) comparing the tissue sample target nucleic acid sequence data with the database target nucleic acid sequence data to determine if the sample target nucleic acid sequence data contains a registered mutation from the mutation database; (c) determining the reliability of the mutation that is registered in the mutation database by determining the mutant allele frequency of the mutation that is registered in the mutation database; and (d) generating a result as to whether the tissue sample target nucleic acid sequence data contains a mutation, thereby detecting the mutation.

In another aspect provided herein is method for determining whether or not a nucleic acid from a preserved tissue sample has a mutation, the method comprising the steps of: a) incubating the preserved tissue sample with a tissue digestion solution to form a tissue digestion mixture; b) heating the tissue digestion mixture at 80 to 110° C. for 1-30 minutes; c) adding a protease solution comprising a proteinase to the tissue digestion mixture to form a protein degradation mixture; d) incubating the protein degradation mixture at 37 to 70° C. for 1-30 minutes; e) incubating the protein degradation mixture at 80 to 110° C. for 1-30 minutes; thereby extracting nucleic acid from the preserved tissue sample; 0 amplifying nucleic acid extracted from the tissue sample, the step of amplification using 5′ phosphorylated oligonucleotides that target a nucleic acid of interest; g) directly ligating an oligonucleotide comprising an adaptor nucleic acid and a barcode nucleic acid to each of the amplified target nucleic acids, thereby making a targeted nucleic acid amplicon library comprising tissue sample target nucleic acid; h) sequencing the library; i) obtaining a tissue sample target nucleic acid sequence data and database target nucleic acid sequence data, wherein the database target nucleic acid sequence data is located in a mutation database; j) comparing the tissue sample target nucleic acid sequence data with the database target nucleic acid sequence data to determine if the sample target nucleic acid sequence data contains a registered mutation from the mutation database; k) determining the reliability of the mutation that is registered in the mutation database by determining the mutant allele frequency of the mutation that is registered in the mutation database; and 1) generating a result as to whether the tissue sample target nucleic acid sequence data contains a mutation, thereby detecting the mutation.

In some embodiments, the tissue digestion solution is selected from i) a tissue digestion solution comprising NaCl at a concentration of 10 mM to 140 mM, Na₂HPO₄ at a concentration of 0.5 mM to 10 mM, KH₂PO₄ at a concentration of 0.1 mM to 5 mM, and Tween 20; ii) a tissue digestion solution comprising NaCl at a concentration of 10 mM to 140 mM, Na₂HPO₄ at a concentration of 0.5 mM to 10 mM, KH₂PO₄ at a concentration of 0.1 mM to 5 mM, and Triton-X100; iii) a tissue digestion solution comprising NaCl at a concentration of 10 mM to 140 mM, Na₂HPO₄ at a concentration of 0.5 mM to 10 mM, and KH₂PO₄ at a concentration of 0.1 mM to 5 mM; iv) a tissue digestion solution comprising TAPS sodium salt at a concentration of 0.5 mM to 25 mM, DTT at a concentration of 0.05 mM to 5 mM, and KCl at a concentration of 0.2 mM to 200 mM; v) a tissue digestion solution comprising HEPES buffer at a concentration of 1 mM to 100 mM; vi) a tissue digestion solution comprising HEPES buffer at a concentration of 1 mM to 100 mM and Triton-X100; vii) a tissue digestion solution comprising HEPES buffer at a concentration of 1 mM to 100 mM and Tween 20; viii) a tissue digestion solution comprising TAPS sodium salt at a concentration of 0.5 mM to 25 mM, DTT at a concentration of 0.05 mM to 5 mM, KCl at a concentration of 0.2 mM to 200 mM, and Triton-X100; ix) a tissue digestion solution comprising a TAPS sodium salt at a concentration of 0.5 mM to 25 mM, DTT at a concentration of 0.05 mM to 5 mM, KCl at a concentration of 0.2 mM to 200 mM, and Tween 20; and x) a tissue digestion solution comprising a TAPS sodium salt at a concentration of 0.5 mM to 25 mM, KCl at a concentration of 0.2 mM to 200 mM, β-Mercaptoethanol at a concentration of 0.1 mM to 1 mM and Triton X-100.

### BRIEF DESCRIPTION OF THE DRAWINGS

FIG. 1 shows the workflow of the nucleic acid extraction procedure provided herein. The method allows for the preparation of genomic DNA from FFPE tissues in a fast, efficient, and cost-effective manner. Unlike some other nucleic acid extraction methods, the method described herein does not involve columns nor toxic chemicals. Only a heat block or a regular thermal cycler (PCR machine) is required for the whole process. The extracted DNA requires no further purification or steps and is ready for the following experiments or genetic analysis (i.e. PCR, qPCR, Sanger Sequencing, NGS, etc).

FIG. 2A and FIG. 2B show that the nucleic acid extraction method provided herein (the “15 min FFPE DNA” method) yields higher amount of genomic DNA compared to that of the QIAGEN QIAmp® DNA FFPE Tissue Kit (A Picogreen quantification). One FFPE slide section (5 μm-thick) from 13 lung adenocarcinoma patients was used for DNA extraction. Two μl of the isolated DNAs, in triplicates, were quantified by Picogreen® method was used to compare the yield of prepared DNA from the 15 min FFPE DNA method and the QIAGEN QIAmp® DNA FFPE Tissue Kit. Red bars indicate the genomic DNA yield from the the 15 min FFPE DNA method and blue bars indicates the genomic DNA yield of the QIAmp® DNA FFPE Tissue Kit (A). The 15 min FFPE DNA kit method produces higher amount of genomic DNA (mean—3.19 fold increase, median—2.13 fold increase) compared to that of the QIAmp® DNA FFPE Tissue Kit (B).

FIG. 3 shows a real-time Quantitative PCR (qPCR) data comparison for the subject nucleic acid extraction method (the “15 min FFPE DNA” method) and the QIAmp® DNA FFPE Tissue Kit. Equal amount of FFPE tissues was used to isolate genomic DNA and eluted in a same volume. Two μl of the isolated DNAs from lung adenocarcinoma FFPE samples (shown in FIG. 2A) were used for qPCR analysis (qPCR probe-RNase Preference gene). Ct (threshold cycle) obtained from the 15 min FFPE DNA method ranges between 21 to 24 cycles while Ct obtained from the QIAmp® DNA FFPE Tissue Kit ranges between 27 to 29 cycles. This shows that DNA from the 15 min FFPE DNA method is more efficiently amplified in qPCR analysis. This result shows that the subject nucleic acid extraction method would be more suitable and ideal for challenging biological specimens with a very low amount tissue or small number of cells.

FIG. 4 shows a workflow of the subject direct amplification and ligation (“NextDay Seq”) amplicon sample library preparation. Ten ng of DNA is amplified using the 5′ phosphorylated oligos and are purified. Barcodes and universal adaptors are directly ligated at 5′ phosphorylated oligos ends. Final purification step provides targeted amplicon libraries ready for template preparation and sequencing. Approximately 2.5 hours are required for the amplicon library preparation.

FIG. 5A and FIG. 5B show a workflow of the whole ‘NextDay Seq’ process. This shows the whole ‘NextDay Seq’ workflow including: FFPE DNA extraction, sample library preparation with 5′-phosphorylated probes and the final sequencing and data analyses. The whole process from DNA extraction to a final data analysis is done within 36 hours. Please note that the first DNA extraction step is performed with the subject nucleic acid extraction method (the “15 min FFPE DNA” method) and the last data analysis step is performed by the subject method (“DanPA”) for detecting a mutation in a target nucleic acid as provided herein.

FIG. 6 shows a general workflow of the subject method for detecting a mutation in a target nucleic acid (Database-associated non-Preprocessing Analysis (DanPA)) for the somatic mutation screening from the NGS sequencing data. This figure shows a general workflow of the DanPA for detecting somatic mutations from the NGS data. DanPA skips almost all known NGS pre/post-processing steps (unmapped sequence re-alignment, dedupping, indel realignment, base quality score recalibration, variant score recalibration, and functional annotation), but detects mutations by directly searching the target sequences in mutation databases. Once the target sequences (i.e. cancer patient DNA sequences) are matched in the mutation databases, the DanPA considers the stability of the registered mutation in the database (i.e. reported time, and homopolymer regions) and checks the mutant allele frequency out of total reads (calculation of the mutant allele frequency). In a case of targeted sequencing with >300 coverage-depth, somatic mutation with 3% of the mutant allele frequency can be robustly detected by DanPA.

FIG. 7 shows a detailed algorithm for the DanPA's workflow. This workflow shows how DanPA compares the patient's (or target DNA) sequences with registered mutations in the designated database (e.g., COSMIC). If the patient's sequences are matched with any registered mutations, DanPA calculates the allele frequency (mutant reads/total reads) and checks the statistical significance for the mutation call. By repeating this step for all amplicons of the targeted sequencing panel, DanPA provides fast and reliable somatic mutation data regardless of mutation type or complexity.

FIG. 8 shows a comparison between the DanPA and the Torrent Suite for somatic mutation detection in lung cancer patients. Two lung cancer patients' somatic mutation analysis results are shown. (A) Although two point mutations (PDGFRA and EGFR-shown in red) were detected by both methods, a deletion mutation of the EGFR gene was detected by only DanPA (blue color). In the 60 lung cancer patients' screening, no single deletion or insertion mutations were detected by Torrent Suite, while all mutations were detected by DanPA. Note that a false-positive (FP) call was detected by Torrent Suite. (B) While four point mutations (shown in red color) were detected by both DanPA and Torrent Suite, one mutation (KIT) with a low allele frequency (around 3%) was detected only by DanPA and missed by Torrent Suite.

FIG. 9 is a block diagram of an electronic network for detecting a mutation in a target nucleic acid sequence

FIG. 10 is a block diagram of the subject device memory shown in FIG. 9, according to some embodiments.

FIG. 11 is a flow chart of a method for detecting a mutation in a target nucleic acid sequence, according to some embodiments.

### DETAILED DESCRIPTION OF THE INVENTION

Provided herein are methods and systems for the detection of genetic mutations from a tissue sample (e.g., a preserved tissue sample). In some embodiments the method includes the steps of a) extracting a nucleic acid from a preserved tissue sample; b) preparing a targeted nucleic acid amplicon library from the extracted nucleic acid; c) sequencing the target nucleic acid amplicon library to produce tissue sample target nucleic acid sequence data; and d) determining whether the target nucleic acid sequence data contains a mutation (e.g., a mutation associated with a risk for a particular disease). The methods described herein advantageously can be performed, from extracting a) to determining d), in less than 48 hours. In certain embodiments, the method can be performed in less than 45, 44, 43, 42, 41, 40, 39, 38, 37, 36, 35, 34, 33, 32, 31, 30, 29, 28, 27, 26, or 25 hours. In certain embodiments, the method can be performed in less than 36 hours. Aspects of the methods and systems provided herein are discussed in detail below.

Nucleic Acid Extraction

In a first aspect, provided herein is a method for extracting a nucleic acid from a tissue sample. In certain embodiments, the method comprises the steps of (a) incubating the tissue sample with a tissue digestion solution to form a tissue digestion mixture; (b) heating the tissue digestion mixture at 80 to 110° C. for 1-30 minutes; (c) adding a proteinase solution comprising a proteinase to the tissue digestion mixture to form a protein degradation mixture and incubating the protein degradation mixture at 50 to 70° C. for 1-30 minutes; and (d) incubating the protein degradation mixture at 80 to 110° C. for 1-30 minutes; thereby extracting nucleic acid from the preserved tissue sample.

The nucleic acid extraction method provided herein provides for a fast and efficient method for extracting nucleic acids from a tissue sample. In some embodiments, the nucleic acid is deoxyribonucleic acid (DNA). In other embodiments, the nucleic acid is ribonucleic acid (RNA). In some embodiments the DNA is genomic DNA. In other embodiments, the DNA is mitochondrial DNA.

Tissue samples that may be used according to the subject methods include, but are not limited to, connective tissue, muscle tissue (e.g., smooth muscle, skeletal muscle, and cardiac muscle), nervous tissue, and epithelial tissue (e.g., squamous epithelium, cuboidal epithelium, columnar epithelium, glandular epithelium, and ciliated epithelium). Tissue samples that may be used according to the subject methods include frozen or fresh tissue samples. In certain embodiments the tissue sample is a preserved tissue sample. As used herein, a “preserved tissue sample” is a tissue sample isolated from a subject that has been subjected to one or more processes to preserve the integrity of the tissue and/or macromolecules (e.g., nucleic acids such as DNA and RNA) of the sample. Techniques for tissue preservation include, but are not limited to, formalin fixation and deep freezing. In some embodiments, the preserved tissue sample is a formalin-fixed, paraffin-embedded (FFPE) tissue sample. FFPE tissue samples may be deparaffinized prior to use with the subject method using any suitable technique, for example, techniques using xylene or a paraffin-solubilizing organic solvent (see, e.g., U.S. Pat. Nos. 6,632,598 and 8,574,868). In certain embodiments, the preserved tissue sample is deparaffinized prior to the incubating in tissue digestion solution (a). In particular embodiments, the preserved tissue sample is deparafiinized in xylene prior to the incubating in tissue digestion solution (a). In some embodiments, the preserved tissue sample is an FFPE that is 1 μm, 2 μm, 3 μm, 4 μm, 5 μm, 6 μm, 7 μm, 8 μm, 9 μm, 10 μm thick.

In certain embodiments, the nucleic acid extraction method can be performed in 90 minutes or less, 60 minutes or less, 55 minutes or less, 50 minutes or less, 45 minutes or less, 40 minutes or less, 35 minutes or less, 30 minutes or less, 25 minutes or less, 20 minutes or less, 15 minutes or less, 14 minutes or less, 13 minutes or less, 12 minutes or less, 11 minutes or less, 10 minutes or less, 9 minutes or less, 8 minutes or less, 7 minutes or less, 6 minutes or less, or 5 minutes or less. In certain embodiments, the nucleic acid extraction method can be performed in 15 minutes or less.

In certain embodiments, the nucleic acid extraction method provided herein includes a first step of incubating the preserved tissue sample with a tissue digestion solution to form a tissue digestion mixture. The tissue digestion solution includes a salt and/or detergent. Salts that can be used in the subject nucleic acid extraction method include, but are not limited to, NaCl, Na₂HPO₄, KH₂PO₄, KCl and TAPS sodium salt. In certain embodiments, the digestion solution comprises NaCl at a concentration of 10 mM to 140 mM. In certain embodiments, the digestion solution comprises Na₂HPO₄ at a concentration of 0.5 mM to 10 mM. In some embodiments, the digestion solution comprises KH₂PO₄ at a concentration of 0.1 mM to 5 mM. In some embodiments, the digestion solution comprises KCl at a concentration of 0.2 mM to 200 mM. In certain embodiments, the digestion solution comprises a TAPS sodium salt at a concentration of 0.5 mM to 25 mM. In certain embodiments, the tissue digestion solution comprises a detergent. Any suitable detergent may be used in the tissue digestion solution. Exemplary detergents that may be used include, but are not limited, Triton-X100 and Tween 20.

In certain embodiments, the tissue digestion solution includes NaCl at a concentration of 10 mM to 140 mM, Na₂HPO4 at a concentration of 0.5 mM to 10 mM, KH₂PO4 at a concentration of 0.1 mM to 5 mM, and Tween 20.

In some embodiments, the tissue digestion solution includes NaCl at a concentration of 10 mM to 140 mM, Na₂HPO4 at a concentration of 0.5 mM to 10 mM, KH₂PO4 at a concentration of 0.1 mM to 5 mM, and Triton-X100.

In some embodiments, the tissue digestion solution includes NaCl at a concentration of 10 mM to 140 mM, Na₂HPO4 at a concentration of 0.5 mM to 10 mM, and KH2PO4 at a concentration of 0.1 mM to 5 mM.

In some embodiments, the tissue digestion solution includes TAPS sodium salt at a concentration of 0.5 mM to 25 mM, DTT at a concentration of 0.05 mM to 5 mM, and KCl at a concentration of 0.2 mM to 200 mM.

In other embodiments, the tissue digestion solution includes HEPES buffer at a concentration of 1 mM to 100 mM.

In some embodiments, the tissue digestion solution includes HEPES buffer at a concentration of 1 mM to 100 mM and Triton-X100.

In other embodiments, the tissue digestion solution includes HEPES buffer at a concentration of 1 mM to 100 mM and Tween 20.

In other embodiments, the tissue digestion solution includes TAPS sodium salt at a concentration of 0.5 mM to 25 mM, DTT at a concentration of 0.05 mM to 5 mM, KCl at a concentration of 0.2 mM to 200 mM, and Triton-X100.

In other embodiments, the tissue digestion solution includes a TAPS sodium salt at a concentration of 0.5 mM to 25 mM, DTT at a concentration of 0.05 mM to 5 mM, KCl at a concentration of 0.2 mM to 200 mM, and Tween 20.

In yet other embodiments, the tissue digestion solution includes a TAPS sodium salt at a concentration of 0.5 mM to 25 mM, KCl at a concentration of 0.2 mM to 200 mM, β-Mercaptoethanol at a concentration of 0.1 mM to 1 mM, and Triton-X100.

In certain embodiments, the tissue digestion mixture is incubated at an optimal temperature and amount of time to promote the digest of the tissue sample. In certain embodiments, the tissue digestion mixture is incubated at a temperature of 60° C., 65° C., 70° C., 75° C., 80° C., 85° C., 90° C., 95° C., 100° C., 105° C., 110° C., 115° C., or 120° C. In some embodiments, the tissue digestion mixture is incubated at a temperature of from 60° C. to 65° C., 65° C. to 70° C., 70° C. to 75° C., 75° C. to 80° C., 80° C. to 85° C., 85° C. to 90° C., 90° C. to 95° C., 95° C. to 100° C., 100° C. to 105° C., 105° C. to 110° C., 110° C. to 115° C., or 115° C. to 120° C. In some embodiments, the tissue digestion mixture is incubated at a temperature from 60° C. to 80° C., 65° C. to 85° C., 70° C. to 90° C., 75° C. to 85° C., 80° C. to 90° C., 85° C. to 95° C., 90° C. to 100° C., 95° C. to 105° C., 100° C. to 110° C., 105° C. to 115° C., or 110° C. to 120° C. In certain embodiments, the tissue digestion mixture is incubated at a temperature from 60° C. to 90° C., 70° C. to 100° C., 80° C. to 110° C. or 90° C. to 120° C. In certain embodiments, the tissue digestion mixture is incubated at a temperature from 80° C. to 110° C. In certain embodiments, the tissue digestion mixture is incubated at 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C. In particular embodiments, the tissue digestion mixture is incubated at 99° C.

In some embodiments, the tissue digestion mixture is incubated for 0.5, 1, 2, 3, 4, 5, 6, 7, 8, 9, 10, 11, 12, 13, 14, 15, 20, 25, 30, 45, or 60 minutes. In certain embodiments, the tissue digestion mixture is incubated for, 1 to 3, 2 to 4, 3 to 5, 4 to 6, 5 to 7, 6 to 8, 7 to 9 or 8 to 10 minutes. In certain embodiments, the tissue digestion mixture is incubated for 1 to 10 minutes, 5 to 15 minutes, 10 to 20 minutes, 15 to 25 minutes, 20 to 30 minutes, 35 to 45 minutes, 40 to 50 minutes, 45 to 55 minutes or 50 to 60 minutes. In particular embodiments, the tissue digestion mixture is incubated for 5 minutes.

In some embodiments, the tissue digestion mixture is incubated at 80° C. to 110° C. for 1 to 30 minutes. In some embodiments, the tissue digestion mixture is incubated at 95° C. to 105° C. for 4 to 6 minutes. In certain embodiments, the tissue digestion mixture is incubated at 99° C. for 5 minutes.

Following the incubation of the tissue digestion mixture, a protease solution comprising a protease is added to the tissue digestion mixture to form a protein degradation mixture. The protein degradation mixture is incubated at a predetermined time and temperature to promote protein degradation. Any protease that aids in the digestion of protein may be included in the proteinase solution of the subject nucleic acid extraction method. Exemplary proteases that may be used include, but are not limited to a serine protease, a threonine protease, a cysteine protease, an aspartate protease, a glutamic acid protease, a metalloprotease or combinations thereof.

In certain embodiments, the protease solution includes a serine protease. Serine proteases are enzymes that cleave peptide bonds in proteins, in which serine serves as the nucleophilic amino acid at the enzyme's active site. Serine proteases include, for example, trypsin-like proteases, chymotrypsin-like proteases, elastase-like proteases and subtilisin-like proteases. Exemplary serine proteases include, but are not limited to, chymotrypsin A, dipeptidase E, subtilisin, nucleoporin, lactoferrin, rhomboid 1 and Proteinase K. In some embodiments, the serine protease is Proteinase K. The predominate site of cleavage of Proteinase K is the peptide bond adjacent to the carboxyl group of aliphatic and aromatic amino acids with blocked alpha amino groups. In certain embodiments, the Proteinase K is present in the protease solution at a concentration of 1 to 100 mg/ml, 2 to 90 mg/ml, 3 to 80 mg/ml, 4 to 70 mg/ml, or 5 to 60 mg/ml. In particular embodiments, the Proteinase K is present in the protease solution at a concentration of 5 to 60 mg/ml. In certain embodiments, the protease solution further comprises a buffer (e.g., Tris-HCl) and/or a protein denaturing agent (e.g., EDTA, UREA or SDS).

In some embodiments, the protease solution includes Proteinase K at a concentration of 5 mg/ml to 60 mg/ml, Tris-HCl at a concentration of 1 mM to 50 mM and EDTA at a concentraiton of 0.1 to 10 mM. In some embodiments, the protease includes Proteinase K at a concentration of 5 mg/ml to 60 mg/ml and Tris-HCl at a concentration of 1 mM to 50 mM. In certain embodiments, the protease includes Proteinase K at a concentration of 5 mg/ml to 60 mg/ml and EDTA at a concentration of 0.1 mM to 10 mM. In certain embodiments, Tris-HCl is at a pH of 8.0

In certain embodiments, the protein degradation mixture is incubated at 30° C., 35° C., 40° C., 45° C., 50° C., 55° C., 60° C., 65° C., 70° C., 75° C., 80° C., 85° C. or 90° C. In some embodiments, the protein degradation mixture is incubated at a temperature from 30° C. to 90° C., 40° C. to 80° C., or 50° C. to 70° C. In some embodiments, the protein degradation mixture is incubated at 30° C. to 35° C., 35° C. to 40° C., 45° C. to 50° C., 55° C. to 60° C., 60° C. to 65° C., 65° C. to 70° C., 70° C. to 75° C., 75° C. to 80° C., 80° C. to 85° C. or 85° C. to 90° C. In particular embodiments, the protein degradation mixture is incubated at 50° C. to 70° C. In certain embodiments, the protein degradation mixture is incubated at 60° C.

In some embodiments, the protein degradation mixture is incubated for at least 0.5, 1, 2, 3, 4, 5, 6, 7, 8, 9, 10, 11, 12, 13, 14, 15, 20, 25, 30, 45, or 60 minutes. In certain embodiments, the protein degradation mixture is incubated for 1 to 3, 2 to 4, 3 to 5, 4 to 6, 5 to 7, 6 to 8, 7 to 9 or 8 to 10 minutes. In certain embodiments, the protein degradation mixture is incubated for 1 to 10 minutes, 5 to 15 minutes, 10 to 20 minutes, 15 to 25 minutes, 20 to 30 minutes, 35 to 45 minutes, 40 to 50 minutes, 45 to 55 minutes or 50 to 60 minutes. In particular embodiments, the protein degradation mixture is incubated for 5 minutes. In certain embodiments, the protein degradation mixture is incubated at 50° C. to 70° C. for 1 to 10 minutes. In certain embodiments, the protein degradation mixture is incubated at 60° C. for 5 minutes.

Following incubation of the protein degradation mixture at a temperature to promote protein degradation, the protein degradation mixture is heated to inactive the protease in the protein degradation mixture, thereby extracting the nucleic acid from the preserved tissue sample. In certain embodiments, the protein degradation mixture is heated to a temperature of 60° C., 65° C., 70° C., 75° C., 80° C., 85° C., 90° C., 95° C., 100° C., 105° C., 110° C. 115° C. or 120° C. to inactivate the protease. In some embodiments, the protein degradation mixture is heated to a temperature of 60° C. to 65° C., 65° C. to 70° C., 70° C. to 75° C., 75° C. to 80° C., 80° C. to 85° C., 85° C. to 90° C., 90° C. to 95° C., 95° C. to 100° C., 100° C. to 105° C., 105° C. to 110° C., 110° C. to 115° C., or 115° C. to 120° C. to inactivate the protease. In some embodiments, the protein degradation mixture is heated to a temperature of 60° C. to 80° C., 65° C. to 85° C., 70° C. to 90° C., 75° C. to 85° C., 80° C. to 90° C., 85° C. to 95° C., 90° C. to 100° C., 95° C. to 105° C., 100° C. to 110° C., 105° C. to 115° C., or 110° C. to 120° C. to inactivate the protease. In certain embodiments, the protein degradation mixture is heated to a temperature of 60° C. to 90° C., 70° C. to 100° C., 80° C. to 110° C. or 90° C. to 120° C. to inactivate the protease. In certain embodiments, the protein degradation mixture is heated to a temperature of 80° C. to 110° C. to inactivate the protease. In certain embodiments, the protein degradation mixture is heated to a temperature of 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C. to inactivate the protease. In particular embodiments, the protein degradation mixture is heated to a temperature of 99° C.

In some embodiments, the protein degradation mixture is incubated for 1, 2, 3, 4, 5, 6, 7, 8, 9, or 10 minutes. In some embodiments, the protein degradation mixture is incubated for 1 to 10 minutes, 5 to 15 minutes, or 10-20 minutes. In particular embodiments, the protein degradation mixture is incubated for 1 to 10 minutes. In certain embodiments, the protein degradation mixture is incubated for 5 minutes. In certain embodiments, the protein degradation mixture is incubated at 80° C. to 110° C. for 5 minutes. In particular embodiments, the protein degradation mixture is incubated at 99° C. for 5 minutes.

Following heating of the protein degradation mixture to denature the protease and extract the nucleic acid, the extracted nucleic acid may be used directly from the protein degradation mixture or may be further isolated and purified by any suitable method known to those skilled in the art, for example, by centrifugation or precipitation (e.g., ethanol precipitation) methods.

Nucleic acid extracted using the subject methods can be used in a wide variety of applications. In certain embodiments, the extracted nucleic acid is DNA that can be directly used (i.e., without further purification after the denaturing of the protease) for polymerase chain reaction (PCR) amplification. In particular, DNA prepared using the subject method can advantageously be used to produce PCR amplicons greater than 900 bp. In some embodiments, the subject nucleic acid extraction method provided herein yields DNA that can produce PCR amplicons that are greater than 900 bp. Such large PCR amplicons can be used, for example, to generate amplicon libraries such as the ones described below.

Targeted Nucleic Acid Amplicon Library

In another aspect, provided herein is a method for making a targeted nucleic acid amplicon library. As used herein, a “targeted nucleic acid amplicon library” refers to a plurality of nucleic acids containing one or more target nucleic acids that have been amplified from a sample (e.g. from nucleic acids extracted from a tissue sample using the subject extraction method) and which can be used for sequencing (e.g., high throughput sequence such as next generation sequencing (NGS)). In some embodiments, the target nucleic acids contain one or more mutant loci associated with a risk for a disease (e.g., a cancer). In some embodiments, the method includes (a) amplifying a nucleic acid extracted from a tissue sample using an oligonucleotide primer pair that targets a nucleic acid of interest (e.g., a nucleic acid that includes one or more mutation loci that is associated with a risk for a disease such as a cancer) to produce targeted nucleic acid amplicons and (b) directly ligating an oligonucleotide comprising an adaptor nucleic acid and/or a bar code nucleic acid to each of the targeted nucleic acid amplicons to make the targeted nucleic acid amplicon library. The subject targeted nucleic acid amplicon library method described herein advantageously provides a quick method for targeted nucleic acid amplicon library construction. In particular, the subject target nucleic amplicon library can be constructed from nucleic acids extracted from a tissue sample in less than 4 hours, in less than 3.5 hours, in less than 3 hours, in less than 2.5 hours, or in less than 2 hours. In certain embodiments, the target nucleic amplicon library can be made in less than 2.5 hours.

In some embodiments, the method includes a first step of amplifying a nucleic acid extracted from a tissue sample using an oligonucleotide primer pair that targets a nucleic acid of interest to produce targeted nucleic acid amplicons. The nucleic acid can be extracted from the tissue sample using any suitable technique including, but not limited to, SDS-Proteinase K, phenol-chloroform, salting out, chromatography based, magnetic bead-base, dendrimer-based or matrix mill nucleic acid extraction techniques. In certain embodiments, the nucleic acid is extracted from the tissue sample using the subject nucleic acid extraction method described herein.

Any target nucleic acid can be targeted for the subject targeted nucleic acid amplicon library production method described herein. In some embodiments, the target nucleic acid is greater than 50 bp, greater than 100 bp, greater than 150 bp, greater than 200 bp, greater than 250 bp, greater than 300 bp, greater than 350 bp, greater than 400 bp, greater than 450 bp, greater than 500 bp, greater than 550 bp, greater than 600 bp, greater than 650 bp, greater than 700 bp, greater than 750 bp, greater than 800 bp, greater than 850 bp, greater than 900 bp, greater than 950 bp, or greater than 1,000 bp long.

In some embodiments the amplifying (a) includes amplifying 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 2,000, 3,000, 4,000, 5,000 or more target nucleic acids of interest.

In certain embodiments, the target nucleic acid of interest includes one or more loci associated with a risk for a disease. In some embodiments, the target nucleic acid includes one or more loci associated with a risk for cancer. Cancer target nucleic acids include, but are not limited to those associated with bladder, brain, breast, colon, liver, ovarian, kidney, lung, renal, colorectal, pancreatic and prostate cancers, as well as cancers of the blood (e.g., leukemia). In certain embodiments, the target nucleic acid is a lung cancer, colorectal cancer and/or pan-cancer (i.e., a collection or combination of multiple cancers) target nucleic acid.

Target nucleic acids may be include one or more loci associated with, but are not limited to, the following diseases: Achondroplasia, Adrenoleukodystrophy, X-Linked, Agammaglobulinemia, X-Linked, Alagille Syndrome, Alpha-Thalassemia X-Linked Mental Retardation Syndrome, Alzheimer Disease, Alzheimer Disease, Early-Onset Familial, Amyotrophic Lateral Sclerosis Overview, Androgen Insensitivity Syndrome, Angelman Syndrome, Ataxia Overview, Hereditary, Ataxia-Telangiectasia, Becker Muscular Dystrophy also The Dystrophinopathies), Beckwith-Wiedemann Syndrome, Beta-Thalassemia, Biotinidase Deficiency, Branchiootorenal Syndrome, BRCA1 and BRCA2 Hereditary CADASIL, Canavan Disease, Cancer, Charcot-Marie-Tooth Hereditary Neuropathy, Charcot-Marie-Tooth Neuropathy Type 1, Charcot-Marie-Tooth Neuropathy Type 2, Charcot-Marie-Tooth Neuropathy Type 4, Charcot-Marie-Tooth Neuropathy Type X, Cockayne Syndrome, Contractural Arachnodactyly, Congenital, Craniosynostosis Syndromes (FGFR-Related), Cystic Fibrosis, Cystinosis, Deafness and Hereditary Hearing Loss, DRPLA (Dentatorubral-Pallidoluysian Atrophy), DiGeorge Syndrome (also 22q11 Deletion Syndrome), Dilated Cardiomyopathy, X-Linked, Down Syndrome (Trisomy 21), Duchenne Muscular Dystrophy (also The Dystrophinopathies), Dystonia, Early-Onset Primary (DYT1), Dystrophinopathies, The Ehlers-Danlos Syndrome, Kyphoscoliotic Form, Ehlers-Danlos Syndrome, Vascular Type, Epidermolysis Bullosa Simplex, Exostoses, Hereditary Multiple, Facioscapulohumeral Muscular Dystrophy, Factor V Leiden Thrombophilia, Familial Adenomatous Polyposis (FAP), Familial Mediterranean Fever, Fragile X Syndrome, Friedreich Ataxia, Frontotemporal Dementia with Parkinsonism-17, Galactosemia, Gaucher Disease, Hemochromatosis, Hereditary, Hemophilia A, Hemophilia B, Hemorrhagic Telangiectasia, Hereditary, Hearing Loss and Deafness, Nonsyndromic, DFNA (Connexin 26), Hearing Loss and Deafness, Nonsyndromic, DFNB 1 (Connexin 26), Hereditary Spastic Paraplegia, Hermansky-Pudlak Syndrome, Hexasaminidase A Deficiency (also Tay-Sachs), Huntington Disease, Hypochondroplasia, Ichthyosis, Congenital, Autosomal Recessive, Incontinentia Pigmenti, Kennedy Disease (also Spinal and Bulbar Muscular Atrophy), Krabbe Disease, Leber Hereditary Optic Neuropathy, Lesch-Nyhan Syndrome Leukemias, Li-Fraumeni Syndrome, Limb-Girdle Muscular Dystrophy, Lipoprotein Lipase Deficiency, Familial, Lissencephaly, Marfan Syndrome, MELAS (Mitochondrial Encephalomyopathy, Lactic Acidosis, and Stroke-Like Episodes), Monosomies, Multiple Endocrine Neoplasia Type 2, Multiple Exostoses, Hereditary Muscular Dystrophy, Congenital, Myotonic Dystrophy, Nephrogenic Diabetes Insipidus, Neurofibromatosis 1, Neurofibromatosis 2, Neuropathy with Liability to Pressure Palsies, Hereditary, Niemann-Pick Disease Type C, Nijmegen Breakage Syndrome Norrie Disease, Oculocutaneous Albinism Type 1, Oculopharyngeal Muscular Dystrophy, Pallister-Hall Syndrome, Parkin Type of Juvenile Parkinson Disease, Pelizaeus-Merzbacher Disease, Pendred Syndrome, Peutz-Jeghers Syndrome Phenylalanine Hydroxylase Deficiency, Prader-Willi Syndrome, PROP 1-Related Combined Pituitary Hormone Deficiency (CPHD), Retinitis Pigmentosa, Retinoblastoma, Rothmund-Thomson Syndrome, Smith-Lemli-Opitz Syndrome, Spastic Paraplegia, Hereditary, Spinal and Bulbar Muscular Atrophy (also Kennedy Disease), Spinal Muscular Atrophy, Spinocerebellar Ataxia Type 1, Spinocerebellar Ataxia Type 2, Spinocerebellar Ataxia Type 3, Spinocerebellar Ataxia Type 6, Spinocerebellar Ataxia Type 7, Stickler Syndrome (Hereditary Arthroophthalmopathy), Tay-Sachs (also GM2 Gangliosidoses), Trisomies, Tuberous Sclerosis Complex. Usher Syndrome Type I, Usher Syndrome Type II, Velocardiofacial Syndrome (also 22q11 Deletion Syndrome), Von Hippel-Lindau Syndrome, Williams Syndrome, Wilson Disease, X-Linked Adrenoleukodystrophy, X-Linked Agammaglobulinemiam X-Linked Dilated Cardiomyopathy (also The Dystrophinopathies), and X-Linked Hypotonic Facies Mental Retardation Syndrome.

In some embodiments, the target nucleic acid includes one or more loci associated with a risk for cancer. Cancer target nucleic acids include, but are not limited to those associated with bladder, brain, breast, colon, liver, kidney, lung, renal, colorectal, pancreatic and prostate cancers, as well as cancers of the blood (e.g., leukemia). In certain embodiments, the target nucleic acid is a lung cancer or colorectal cancer or pan-cancer target nucleic acid. In some embodiments, the amplifying a nucleic acid extracted from a tissue sample (a) is performed using one or more of the oligonucleotide primer pairs disclosed in Table 1, Table 2 or Table 3 below. Tables 1, 2, and 3 provide primer pair panels that are useful for the preparation of amplicon library of target nucleic acids containing loci associated with lung cancer, colorectal cancer, and more than one type of cancer (i.e., a “pan-cancer” panel), respectively. In certain embodiments, each of the oligonucleotides of the oligonucleotide primer pair comprises a phosphorylated 5′end. Oligonucleotide primer pairs with phosphorylated 5′ ends advantageously allow for the direct ligation of oligonucleotides to the targeted nucleic acid amplicons, barcode oligonucleotides, adaptor oligonucleotides or combinations thereof. Exemplary oligonucleotides that can be ligated to the 5′ ends of the targeted nucleic acid amplicons include oligonucleotides that include or more elements to facilitate sequencing of the targeted nucleic acid amplicons (e.g., bar codes and universal adaptors).

In certain embodiments, the subject method for making a targeted nucleic acid amplicon library includes a step of purifying the amplified target nucleic acids amplicons prior to ligation of an oligonucleotide to the phosphorylated 5′ end of each of the targeted nucleic acid amplicons. Any suitable technique can be used to purify the amplified targeted nucleic acid amplicon include ethanol/isopropanol precipitation and filtration/affinity column techniques.

In some embodiments, the method further comprises the step of directly ligating an oligonucleotide comprising an adaptor nucleic acid and/or a barcode nucleic acid to each phosphorylated 5′ end of the amplified target nucleic acids, thereby making a targeted nucleic acid amplicon library. As used herein, “directly ligate”, “direct ligation” and the like refer to the process of ligation of oligonucleotides in the absence of an enzyme or preparation of the 5′ ends (e.g., end-polishing) of the amplified target nucleic acids for ligation. In certain embodiments, the step of directly ligating includes the ligation of an oligonucleotide comprising an adaptor nucleic acid to each phosphorylated 5′ end of the amplified target nucleic acids. As used herein an “adaptor nucleic acid” is an oligonucleotide containing a nucleic acid sequence that allow for the clonal amplification of a particular targeted nucleic acid amplicon, for example, by emulsion PCR. In certain embodiments, the adaptor sequence is complementary to that of an oligonucleotide attached to a bead used in emulsion PCR. In certain embodiments, the adaptor sequence is 3, 4, 5, 6, 7, 8, 9, 10, 11, 12, 13, 14, 15, 16, 17, 18, 19, 20, 25, 30, 35, 40 nucleotides in length. In other embodiments, the step of directly ligating includes the ligation of an oligonucleotide comprising a barcode nucleic acid to each phosphorylated 5′ end of the amplified target nucleic acids. As used herein a “barcode sequence” is a nucleic acid sequence that allow for targeted nucleic acid amplicons from different samples (e.g. different tissue samples) to be distinguished from one another during sequencing of pooled targeted nucleic acid amplicon libraries (e.g., multiplex sequencing, see, e.g., Smith et al. Nucleic Acids Res., 38(13): e142 (2010)). In certain embodiments, the barcode sequence is 3, 4, 5, 6, 7, 8, 9, 10, 11, 12, 13, 14, 15, 16, 17, 18, 19, 20, 25, 30, 35, 40 nucleotides in length. In yet other embodiments, the step of directly ligating includes the ligation of an oligonucleotide comprising an adaptor nucleic acid and a barcode nucleic acid to each phosphorylated 5′ end of the amplified target nucleic acids.

Following construction of the targeted nucleic acid amplicon library, the library may be sequenced using any method known in the art to produce target nucleic acid sequence data. In certain embodiments, the targeted nucleic acid amplicon library is sequenced using any Next Generation Sequencing (NGS) method known in the art. NGS sequencing methods include, but are not limited to, single-molecule real-time sequencing (e.g., Pacific Bio), ion semiconductor methods (Ion Torrent sequencing), pyrosequencing (e.g., 454 Life Sciences), sequencing by synthesis (e.g., Illumina sequencing and single molecule real time (e.g., SMRT) sequencing), sequencing by ligation (e.g., SOLiD sequencing), chain termination sequencing (e.g., Sanger sequencing), bead based sequencing (e.g., massively parallel signature sequencing (MPSS)), polony sequencing, DNA nanoball sequencing, heliscope single molecule sequencing (e.g., Heilscope Biosciences).

Genetic Mutation Analysis

Following sequencing of the targeted nucleic acid amplicon library, the target nucleic acid sequences can be subjected to analysis for the detection of a genetic mutation. In another aspect provided herein is a method for detecting a mutation in a tissue sample target nucleic acid sequence, the method comprising a) obtaining a tissue sample target nucleic acid sequence data and database target nucleic acid sequence data, wherein the database target nucleic acid sequence data is located in a mutation database; b) comparing the tissue sample target nucleic acid sequence data against the database target nucleic acid sequence data to determine if the sample target nucleic acid sequence data contains a registered mutation from the mutation database; c) determining the reliability of the mutation that is registered in the mutation database by determining the mutant allele frequency of the mutation that is registered in the mutation database; and d) generating a result as to whether the tissue sample target nucleic acid sequence data contains a mutation, thereby detecting the mutation.

The subject method for detection of a mutation can be used to determine any type of genetic mutation. In certain embodiments, the method is used to detect a point mutation, a deletion, an insertion, an amplification or any other mutation that is registered in a genetic mutation database. In some embodiments, the method is for the detection of a genetic mutation that is registered in the Catalogue of Somatic Mutations in Cancer (COSMIC, http://cancer.sanger.ac.uk/cancergenome/projects/cosmic/), ClinVar (http://www.ncbi.nlm.nih.gov/clinvar/) and/or Online Mendelian Inheritance in Man (OMIM, http://www.omim.org) and/or any variation (mutation) database.

In certain embodiments, the tissue sample target nucleic acid sequence data used in the subject method for detection is data that has not been preprocessed. As used herein “preprocessed data” refers to data that has been subjected to unmapped sequence re-alignment, de-duplication of data processing, indel realignment, base quality score calibration, variant score recalibration and/or functional annotation. In certain embodiments, the comparing b) is performed using tissue sample target nucleic acid sequence data that has not been preprocessed.

In certain embodiments, the subject method allows for the detection of a mutation in a tissue sample target nucleic acid sequence in less than 2 days, 1 day, 12 hours, 6 hours, 5 hours, in less than 4 hours, in less than 3 hours, in less than 2 hours, in less than 1 hour, or in less than 30 minutes. In particular embodiments, the subject method allows for the detection of a mutation in a tissue sample target nucleic acid sequence in less than one hour.

In another aspect provided herein is a computing system that includes one or more processors, memory and one more programs, wherein the one or more programs are stored in the memory and are configured to be executed by the one or more processors for detecting a mutation in a tissue sample target nucleic acid sequence, wherein the one or more programs include instructions for detecting a mutation in a tissue sample target nucleic acid sequence comprising: a) obtaining a tissue sample target nucleic acid sequence data and database target nucleic acid sequence data, wherein the database target nucleic acid sequence data is located in a mutation database; b) comparing the tissue sample target nucleic acid sequence data against the database target nucleic acid sequence data to determine if the sample target nucleic acid sequence data contains a registered mutation from the mutation database; c) determining the reliability of the mutation that is registered in the mutation database by determining the mutant allele frequency of the mutation that is registered in the mutation database; and d) generating a result as to whether the tissue sample target nucleic acid sequence data contains a mutation, thereby detecting the mutation.

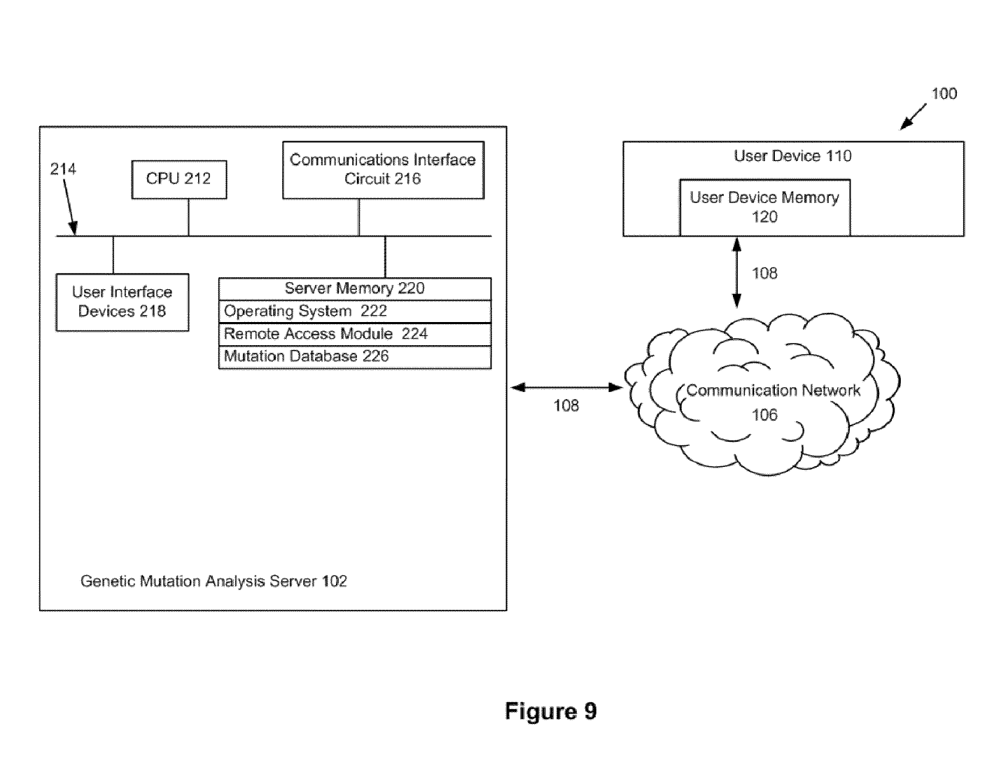

FIG. 9 is a diagrammatic view of an electronic network 100 for the detection of a genetic mutation with some embodiments. The network 100 comprises a series of points or nodes interconnected by communication paths. The network 100 may interconnect with other networks, may contain subnetworks, and may be embodied by way of a local area network (LAN), a metropolitan area network (MAN), a wide area network (WAN), or a global network (the Internet). In addition, the network 100 may be characterized by the type of protocols used on it, such as WAP (Wireless Application Protocol), TCP/IP (Transmission Control Protocol/Internet Protocol), NetBEUI (NetBIOS Extended User Interface), or IPX/SPX (Internetwork Packet Exchange/Sequenced Packet Exchange). Additionally, the network 100 may be characterized by whether it carries voice, data, or both kinds of signals; by who can use the network 100 (whether it is public or private); and by the usual nature of its connections (e.g. dial-up, dedicated, switched, non-switched, or virtual connections).

The network 100 connects a plurality of user devices 110 to at least one genetic mutation analysis server 102. This connection is made via a communication or electronic network 106 that may comprise an Intranet, wireless network, cellular data network or preferably the Internet. The connection is made via communication links 108, which may, for example, be coaxial cable, copper wire (including, but not limited to, PSTN, ISDN, and DSL), optical fiber, wireless, microwave, or satellite links. Communication between the devices and servers preferably occurs via Internet protocol (IP) or an optionally secure synchronization protocol, but may alternatively occur via electronic mail (email).

The genetic mutation analysis server 102 is shown in FIG. 9, and is described below as being distinct from the user devices 110. The genetic mutation analysis server 102 comprises at least one data processor or central processing unit (CPU) 212, a server memory 220, (optional) user interface devices 218, a communications interface circuit 216, and at least one bus 214 that interconnects these elements. The server memory 220 includes an operating system 222 that stores instructions for communicating, processing data, accessing data, storing data, searching data, etc. The server memory 220 also includes remote access module 224 and a mutation database 226. In some embodiments, the remote access module 224 is used for communicating (transmitting and receiving) data between the genetic mutation analysis server 102 and the communication network 106. In some embodiments, the mutation database 226 is used to store mutation database target nucleic acid sequence data that includes registered genetic mutations and that can be used by one or more programs of the computing system provided herein (e.g., programs for detecting a genetic mutation). In certain embodiments, the mutation database 226 includes mutation database target nucleic acid sequence data containing registered genetic mutations that are associated with a particular disease. In some embodiments the genetic mutation database includes genetic mutations that are registered in the Catalogue of Somatic Mutations in Cancer (COSMIC), ClinVar and/or OMIM and/or any variation (mutation) database.

In some embodiments, a user device 110 is a device used by a user who is determining whether or not a target nucleic acid has a mutation (e.g., a mutation associated with a disease). The user device 110 accesses the communication network 106 via remote client computing devices, such as desktop computers, laptop computers, notebook computers, handheld computers, tablet computers, smart phones, or the like. In some embodiments, the user device 110 includes a data processor or central processing unit (CPU), a user interface device, communications interface circuits, and buses, similar to those described in relation to the genetic mutation analysis server 102. The subject device 110 also includes memories 120, described below. Memories 220 and 120 may include both volatile memory, such as random access memory (RAM), and non-volatile memory, such as a hard-disk or flash memory.

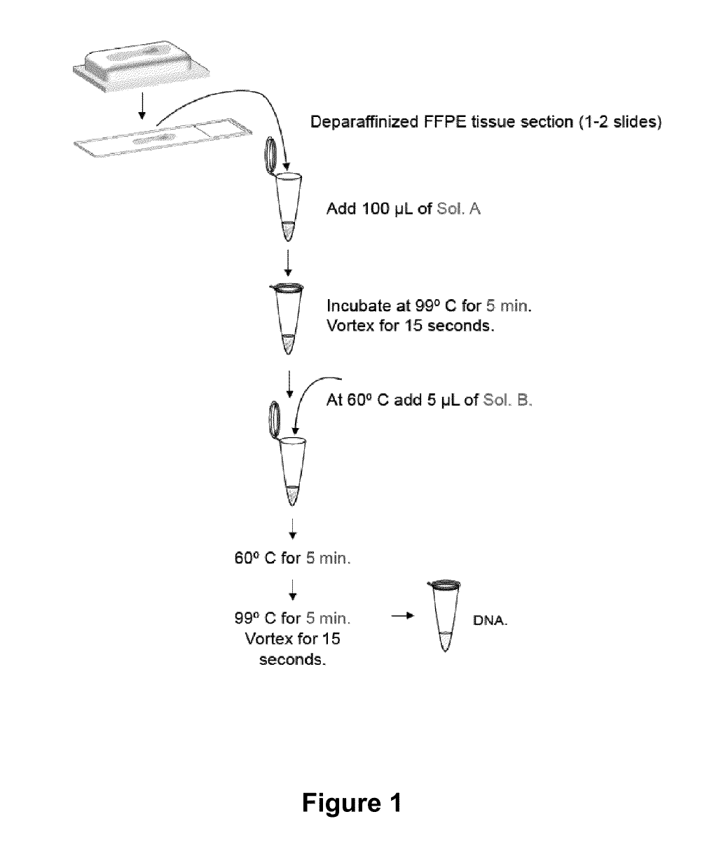

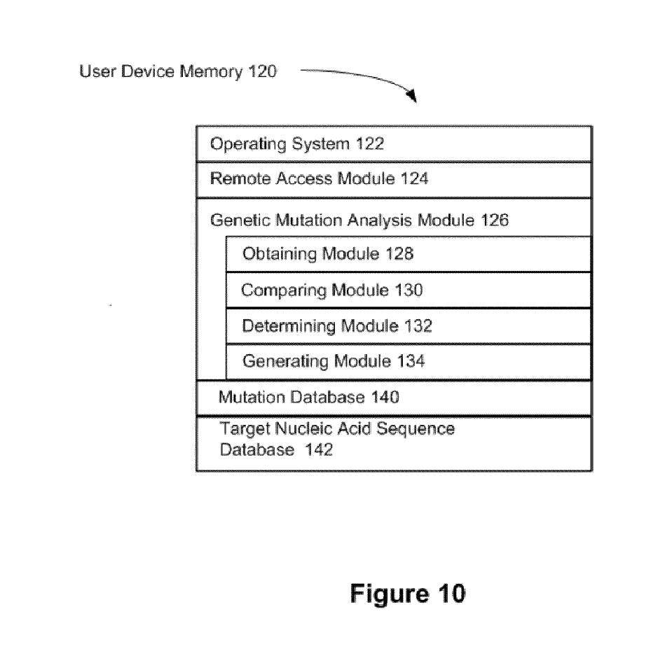

FIG. 10 is a block diagram of a user device memory 120 shown in FIG. 9, according to some embodiments. The subject device memory 120 includes an operating system 122 and remote access module 124 compatible with the remote access module 224 (FIG. 1) in the server memory 220 (FIG. 1).

In some embodiments, the user device memory 120 includes a genetic mutation analysis module 126. The genetic mutation analysis module 126 includes instructions for detecting a genetic mutation in a target nucleic acid sequence, as detailed below. In some embodiments, the genetic mutation analysis module 126 comprises one or more modules for detecting a genetic mutation in a target nucleic acid sequence. For instance, in some embodiments, the genetic mutation analysis module 126 included in the user device memory 120 comprises an obtaining module 128, a comparing module 130, a determining module 132, and a generating module 134.

In some embodiments, the user device memory 120 also comprises a mutation database 140. In certain embodiments, the mutation database 140 comprises mutation database target nucleic acid sequence data containing registered genetic mutations that are associated with a particular disease and that are used in the method of detection of the computing system as described below. In some embodiments the genetic mutation database includes the genetic mutations that are registered in the Catalogue of Somatic Mutations in Cancer (COSMIC), ClinVar and/or OMIM and/or any variation (mutation) database.

In some embodiments, the user device memory 120 also includes a sample target nucleic acid sequence database 142. In some embodiments, the sample target nucleic acid sequence database contains target nucleic acid sequence data obtained from preserved tissue samples using the subject methods described herein.

It should be noted that the various databases described above have their data organized in a manner so that their contents can easily be accessed, managed, and updated. The databases may, for example, comprise flat-file databases (a database that takes the form of a table, where only one table can be used for each database), relational databases (a tabular database in which data is defined so that it can be reorganized and accessed in a number of different ways), or object-oriented databases (a database that is congruent, with the data defined in object classes and subclasses). The databases may be hosted on a single server or distributed over multiple servers. In some embodiments, there is a mutation database 226 but no mutation database 140.

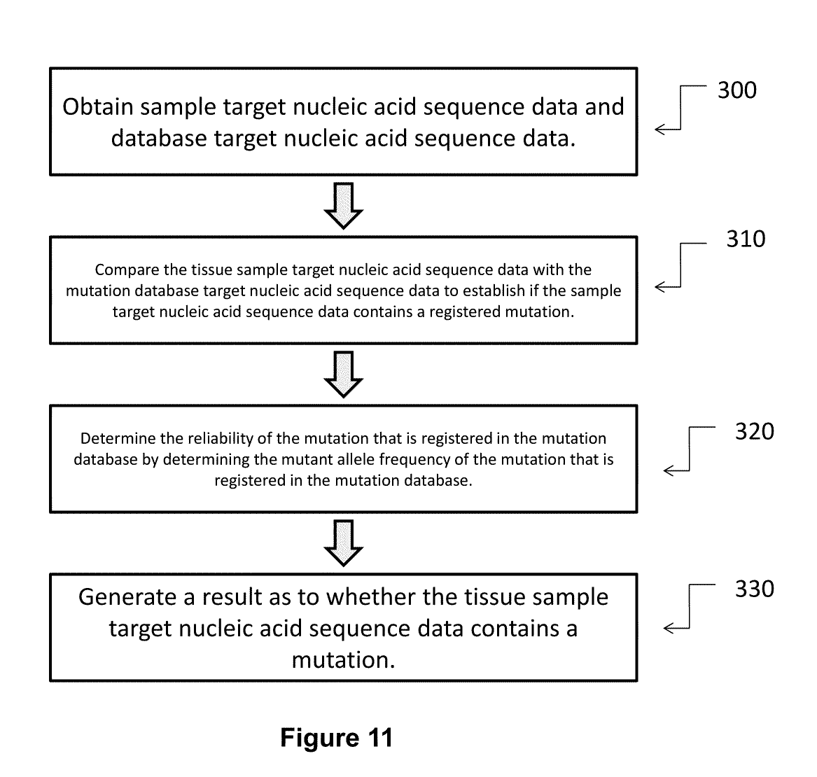

FIG. 11 is a flow chart that illustrates the method 300 for the detection of a mutation in a target nucleic acid (e.g., one obtained and amplified from a preserved tissue sample using the methods described herein), according to some embodiments of the subject computing system. In some embodiments, the method is carried out by one or more programs of the subject computer system described herein.

In some embodiments, the method comprises (a) obtaining sample target nucleic acid sequence data and mutation database target nucleic acid sequence data 300; (b) comparing the tissue sample target nucleic acid sequence data with the mutation database target nucleic acid sequence data to establish if the sample target nucleic acid sequence data contains a registered mutation 310; (c) determining the reliability of the mutation that is registered in the mutation database by determining the mutant allele frequency of the mutation that is registered in the mutation database 320; and (d) generating a result as to whether the tissue sample target nucleic acid sequence data contains a mutation, thereby detecting the mutation 330.

In some embodiments, the method for detecting a mutation in a target nucleic acid comprises obtaining sample target nucleic acid sequence data and mutation database target nucleic acid sequence data 300. In certain embodiments of the computing system provided herein, the obtaining (a) is performed according to instructions included in the obtaining module 128 stored in the user device memory 120 of a user device 110. In certain embodiments, the mutation database target nucleic acid sequence data is obtained from a mutation database 226 that is stored in the server memory 220 of a genetic mutation analysis server 102. In certain embodiments, the mutation database target nucleic acid sequence data is obtained from a mutation database 140 that is stored in the user device memory 120 of a user device 110. As used herein “mutation database target nucleic acid sequence data” refers to any nucleic acid sequence data relating to a particular target nucleic acid that is stored in a mutation database. Exemplary mutation databases include, but are not limited to, Catalogue of Somatic Mutations in Cancer (COSMIC), ClinVar and Online Mendelian Inheritance in Man (OMIM, http://www.omim.org). In certain embodiments, the mutation database 140 or 226 contains mutations that are associated with a particular disease. In some embodiments the genetic mutation database includes the genetic mutations that are registered in the Catalogue of Somatic Mutations in Cancer (COSMIC). In certain embodiments, the sample target nucleic acid sequence data has not been subjected to unmapped sequence re-alignment, de-deplication, indel realignment, base quality score calibration, variant score recalibration and/or functional annotation (i.e., has not been subjected to preprocessing).

In certain embodiments, following the obtaining (a) 300, the method comprises the step of comparing the tissue sample target nucleic acid sequence data with the mutation database target nucleic acid sequence data to establish if the sample target nucleic acid sequence data contains a registered mutation 310. In certain embodiments of the computing system provided herein, the comparing (b) is performed according to instructions included in a comparing module 130 stored in the user device memory 120 of a user device 110. In some embodiments, the tissue sample target nucleic acid sequence data is compared with 10 or more, 20 or more, 30 or more, 40 or more, 50 or more, 60 or more, 70 or more, 80 or more, 90 or more, 100 or more, 150 or more, 200 or more, 250 or more, 300 or more, 350 or more, 400 or more, 450 or more, 500 or more, 600 or more, 700 or more, 800 or more, 900 or more individual mutation database target nucleic acid sequence “reads” in the genetic mutation database 140 or 226 to determine if the sample target nucleic acid sequence data contains a mutation that is a registered mutation in the genetic mutation database 140 or 226.

If the sample target nucleic acid sequence data is deemed to contain a mutation that is a registered mutation in the mutation database 140 or 226, the reliability of the registered mutation is further determined. In certain embodiments, the method comprises (c) determining the reliability of the mutation that is registered in the mutation database by determining the mutant allele frequency of the mutation that is registered in the mutation database 320. In certain embodiments of the computing system provided herein, the determining (c) is performed according to instructions included in a determining module 132 stored in the user device memory 120 of a user device 110. In certain embodiments, the registered mutation is determined to be reliable if it is present above a threshold mutant allele frequency. In some embodiments, the registered mutation is determined to be reliable if it is present above a threshold percentage of the total mutation database target nucleic acid sequence “reads” in the comparing (b) 310. In some embodiments, the registered mutation is determined to be reliable if it is present above 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, or 70% of the total mutation database target nucleic acid sequence “reads” in the comparing (b). In certain embodiments, the determining module 132 determines whether the registered mutation is reliable by counting the number of mutation database target nucleic acid sequence “reads” that contain the registered mutation, selecting an algorithm in static models, determining a P-value, and filtering in results.

In certain embodiments, the method includes the step of (d) generating a result as to whether the tissue sample target nucleic acid sequence data contains a mutation and thereby detecting the mutation 330 following the determining (c). In certain embodiments of the computing system provided herein, the generating (d) is performed according to instructions included in a generating module 134 stored in the user device memory 120 of a user device 110.

### EXAMPLES

### Example 1

### Nucleic Acid Extraction Method

A fast and simple method of nucleic acid extraction, in particular DNA, was developed to maximize the yield and quality of the minimum amount of FFPE tissue. Particular, the nucleic acid extraction method allows for the extraction of nucleic acids in 15 minutes or less (the “15 min FFPE DNA kit”). Further, unlike most other commercial FFPE nucleic extraction methods, the new method uses neither column nor specialized material except two solutions (Solutions A and B).

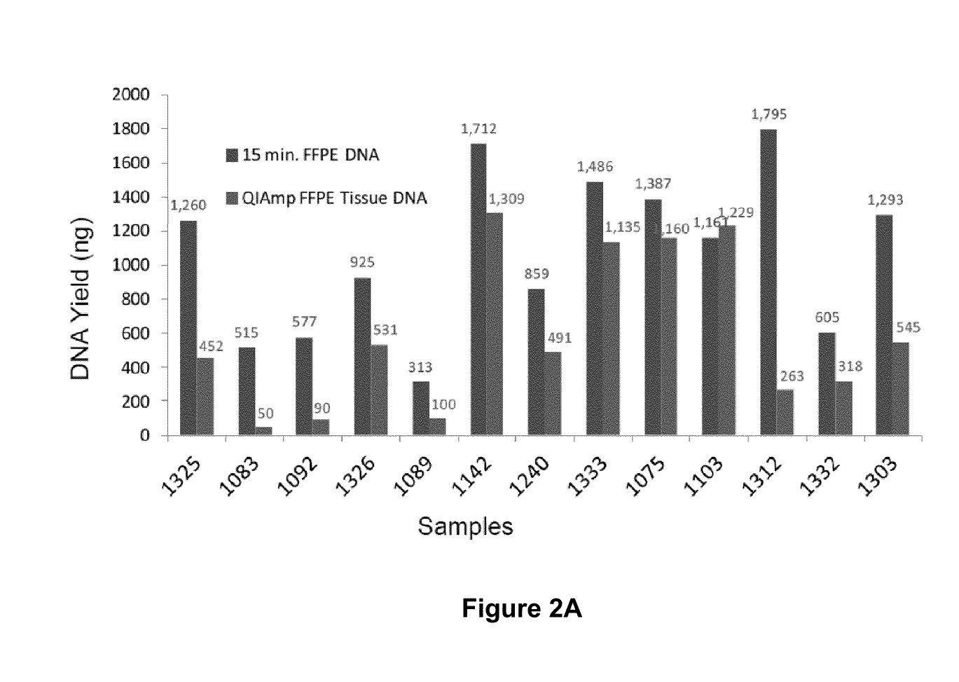

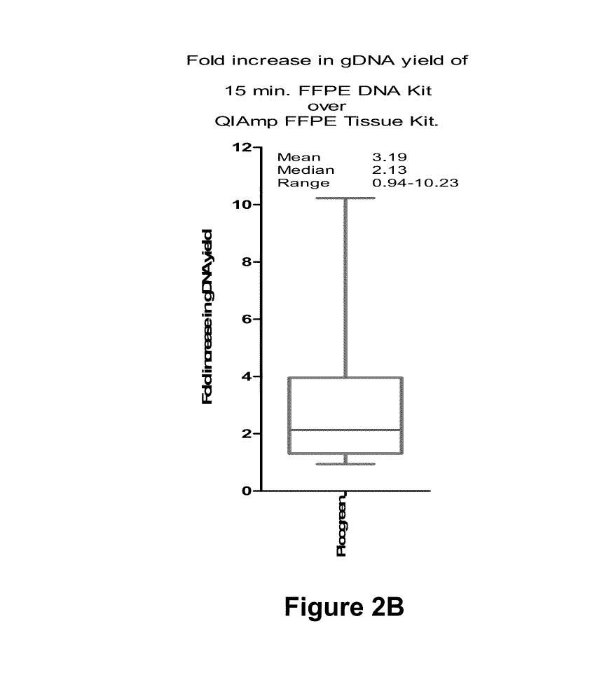

As shown in a general workflow in FIG. 1, this method can be used in any laboratory or facility equipped with a simple heat block or a regular thermal cycler. Deparaffinized FFPE tissue sections are incubated with the solution A at 99° C. for 5 minutes and then with solution B at 60° C. for another 5 minutes. A final incubation at 99° C. for 5 minutes produces a high yield and quality of DNA. FIG. 2 shows that that the nucleic acid extraction method provided yielded higher amounts of DNA as compared to the market leading QIAGEN QIAmp® DNA FFPE Tissue Kit. One FFPE slide section (5 μm-thick) each from 13 lung adenocarcinoma patients was used for DNA extraction. A Picogreen® method was used for quantitating DNA prepared from ‘15 min FFPE DNA kit’ and the QIAGEN QIAmp® DNA FFPE Tissue Kit. Red bars indicate the genomic DNA yield from the ‘15 min FFPE DNA kit’ and blue bars indicates the genomic DNA yield of the QIAmp® DNA FFPE Tissue Kit (A). The ‘15 min FFPE DNA kit’ produces higher amount of genomic DNA (mean—3.19 fold increase, median—2.13 fold increase) compared to that of the QIAmp® DNA FFPE Tissue Kit (B).

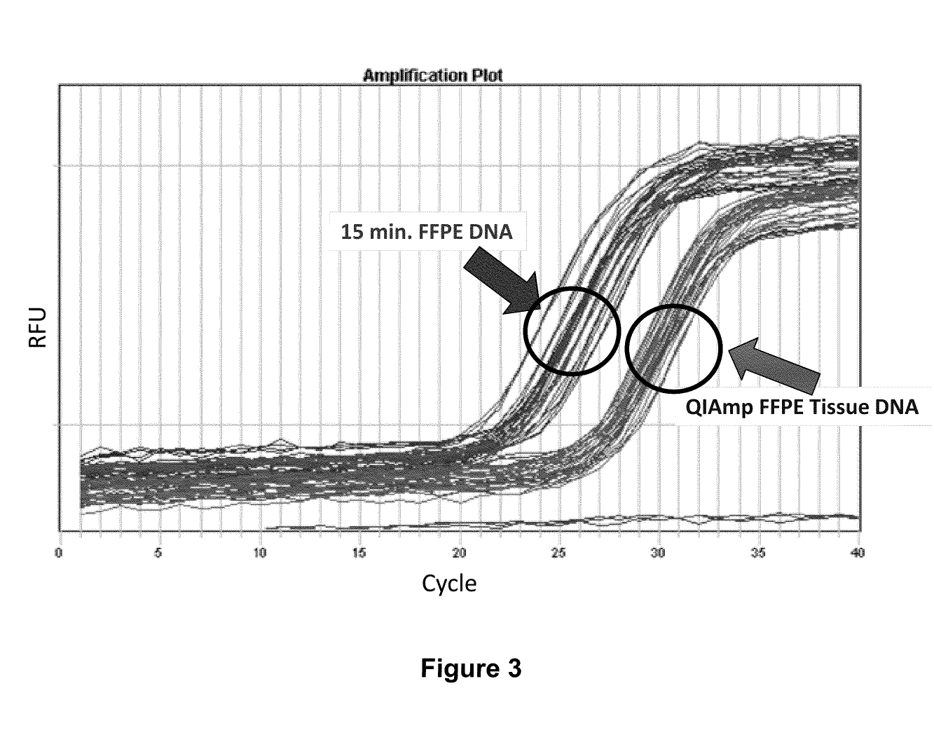

FIG. 3 demonstrates that the nucleic acid extracted from the 15 min FFPE DNA kit can be used for any PCR-based (i.e. quantitative PCR (qPCR), Sanger sequencing, and next-generation sequencing) or genetic analysis. Equal amount of FFPE tissues was used to isolate genomic DNA and eluted in a same volume. Two μl of the isolated DNAs from lung adenocarcinoma FFPE samples (shown in FIG. 2A) were used for qPCR analysis (qPCR probe-RNase Preference gene). Ct (threshold cycle) obtained from the 15 min FFPE DNA kit′ ranged between 21 to 24 cycles while Ct obtained from the QIAmp® DNA FFPE Tissue Kit ranges between 27 to 29 cycles. This showed that DNA from the 15 min FFPE DNA kit was more efficiently amplified in qPCR analysis.

From only one 5 μm-thick FFPE slide, up to 2 ug of DNA can be obtained. The method is also very efficient for qPCR and large-size PCR (more than 1 kb) analyses. Unlike most other known and commercial methods, the ‘15 min FFPE DNA kit’ enables large amplicon analysis, which makes FFPE sample analysis more flexible and applicable in the clinical genetic analysis.

### Example 2

### Nucleic Acid Amplicon Preparation Method

A simple, and robust sample amplicon preparation method called ‘NextDay Seq,’ was developed to enable the obtaining of targeted deep sequencing data within the next day of sample arrival. In short, researchers and medical doctors can obtain sequencing data within 36 hours, starting DNA extraction from a given sample (i.e. Formalinfixed, paraffin-embedded (FFPE) tissue samples), library preparation, sequencing and data analysis.

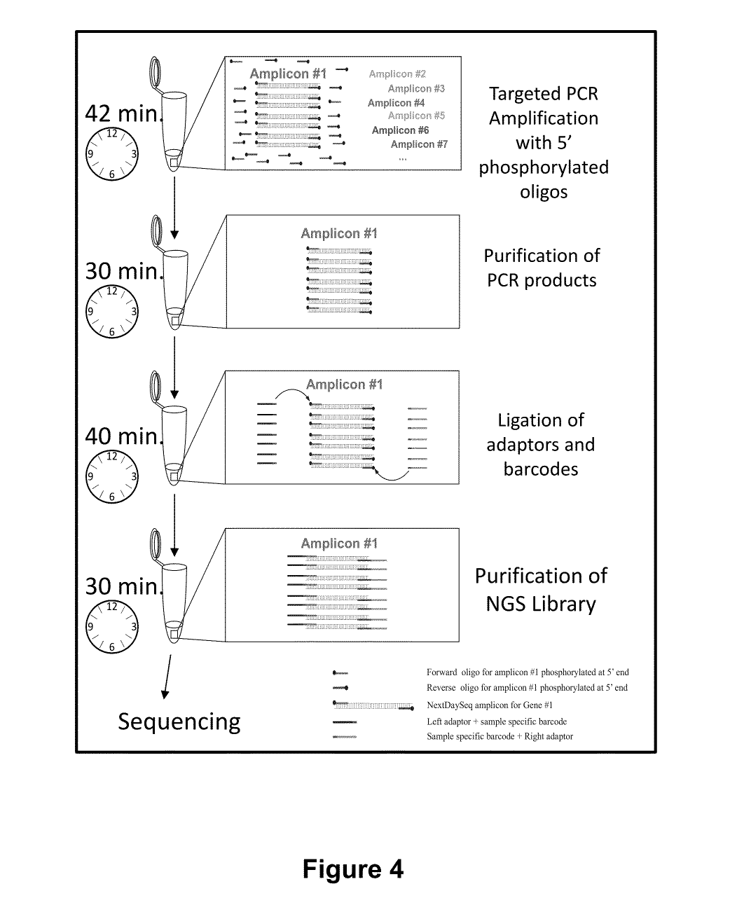

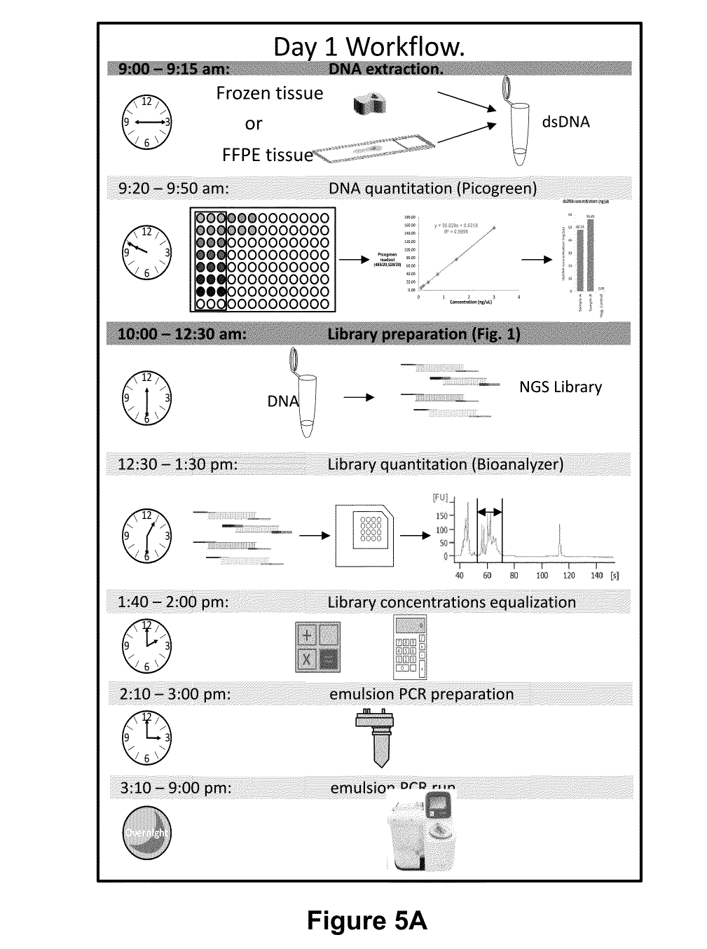

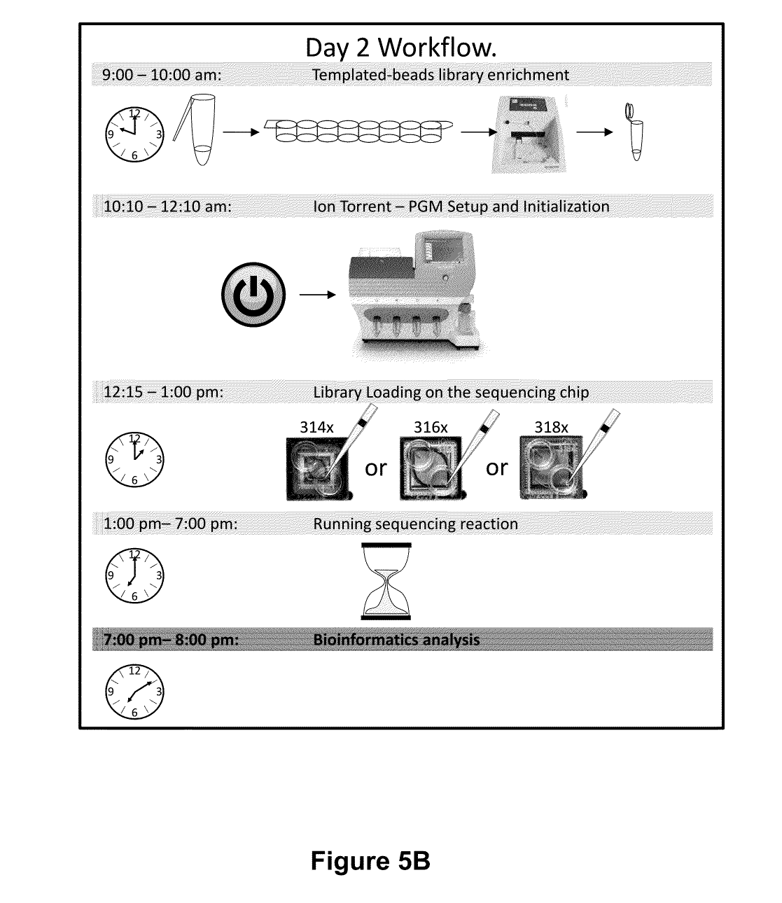

Here, a direct ligation method with the multiplex amplification of the target genes or amplicons by using 5′-phosphorylated oligonucleotides (FIGS. 4 and 5). This protocol does not require an enzyme digestion or hybridization of the target region. For use in the direct amplification and ligation method described herein, targeted NGS panels were developed that designing probe sequences targeting commonly mutated genes as therapeutic foci in the human lung (Table 1), colorectal (Table 2), and pan cancers. Further, such amplicon preparation method can be applied to any cancer or gene panel by modifying probe sequences targeting genes of interest.

TABLE 1
5′ Phosphorylated Oligonucleotide
Sequences for The Lung Cancer Panel.
(SEQ ID NO: 1-SEQ ID NO: 66)

| Amplicon | | Gene |
|---|---|---|
| ID | Oligo Sequence 5′-3′ | Symbol |
| LU-4 | GGGTGAGGCAGTCTTTACTCAC | ALK_FW |
| LU-4 | GCCGTTGTACACTCATCTTCCTAG | ALK_RV |
| LU-5 | CCAATGCAGCGAACAATGTTCTG | ALK_FW |
| LU-5 | TGCCTTTATACATTGTAGCTGCTGAAA | ALK_RV |
| LU-21 | ACAACAACTGCAGCAAAGACTG | ALK_FW |
| LU-21 | GCTCTGCAGCAAATTCAACCAC | ALK_RV |
| LU-22 | GGGTGTCTCTCTGTGGCTTTAC | ALK_FW |
| LU-22 | CTCTGTAGGCTGCAGTTCTCAG | ALK_RV |
| LU-16 | ACTCCATCGAGATTTCACTGTAGCTA | BRAF_FW |
| LU-16 | TCTCTTACCTAAACTCTTCATAATGCTTGC | BRAF_RV |
| LU-17 | CATACTTACCATGCCACTTTCCCTT | BRAF_FW |
| LU-17 | CTTTTTCTGTTTGGCTTGACTTGACTT | BRAF_RV |
| LU-32 | AATGACTTTCTAGTAACTCAGCAGCAT | BRAF_FW |
| LU-32 | CCTCACAGTAAAAATAGGTGATTTTGGTC | BRAF_RV |
| LU-33 | CCTATTATGACTTGTCACAATGTCACCA | BRAF_FW |
| LU-33 | TAGACGGGACTCGAGTGATGATT | BRAF_RV |
| LU-1 | GGGTTACTTTGTGGGAGACTTTCA | DDR2_FW |
| LU-1 | ACAGGTCCACATCCATTCATCC | DDR2_RV |
| LU-18 | TAGCTGCAGATTATGAAATTTAACAGGGT | DDR2_FW |
| LU-18 | GAATAGGGCTGTTCTTGACAAAAGG | DDR2_RV |
| LU-11 | GGTGACCCTTGTCTCTGTGTTC | EGFR_FW |
| LU-11 | AGGGACCTTACCTTATACACCGT | EGFR_RV |
| LU-12 | CTGGTAACATCCACCCAGATCA | EGFR_FW |
| LU-12 | GGAGATGTTGCTTCTCTTAATTCCTTG | EGFR_RV |
| LU-13 | TCTGGCCACCATGCGAAGC | EGFR_FW |
| LU-13 | GGCATGAGCTGCGTGATGA | EGFR_RV |
| LU-14 | GGACTATGTCCGGGAACACAAA | EGFR_FW |
| LU-14 | ATGGCAAACTCTTGCTATCCCA | EGFR_RV |
| LU-15 | TGTCAAGATCACAGATTTTGGGCT | EGFR_FW |
| LU-15 | ATGTGTTAAACAATACAGCTAGTGGGAA | EGFR_RV |
| LU-28 | GGAAACTGAATTCAAAAAGATCAAAGTGCT | EGFR_FW |
| LU-28 | GGAAATATACAGCTTGCAAGGACTCT | EGFR_RV |
| LU-29 | TGAGAAAGTTAAAATTCCCGTCGCTAT | EGFR_FW |
| LU-29 | CTGCCAGACATGAGAAAAGGTG | EGFR_RV |
| LU-30 | GAAGCCTACGTGATGGCCA | EGFR_FW |
| LU-30 | CAGGTACTGGGAGCCAATATTGTC | EGFR_RV |
| LU-31 | CACAGCAGGGTCTTCTCTGTTT | EGFR_FW |
| LU-31 | CCTTCTGCATGGTATTCTTTCTCTTCC | EGFR_RV |
| LU-2 | TGTCAGCTTATTATATTCAATTTAAACCCACCT | KRAS_FW |
| LU-2 | CAGGTCAAGAGGAGTACAGTGC | KRAS_RV |
| LU-3 | CAAAGAATGGTCCTGCACCAGTA | KRAS_FW |
| LU-3 | AAGGCCTGCTGAAAATGACTGAATATA | KRAS_RV |
| LU-19 | TCCTCATGTACTGGTCCCTCATT | KRAS_FW |
| LU-19 | GGTGCACTGTAATAATCCAGACTGT | KRAS_RV |
| LU-20 | GCTGTATCGTCAAGGCACTCTTG | KRAS_FW |
| LU-20 | AGGTACTGGTGGAGTATTTGATAGTGTATT | KRAS_RV |
| LU-8 | GGTGCACTGGGACTTTGGTAAT | PDGFRA_FW |
| LU-8 | TCCATCTCTTGGAAACTCCCATCT | PDGFRA_RV |
| LU-9 | TCTGAGAACAGGAAGTTGGTAGCT | PDGFRA_FW |
| LU-9 | CAGCAAGTTTACAATGTTCAAATGTGG | PDGFRA_RV |
| LU-10 | GGGTGATGCTATTCAGCTACAGA | PDGFRA_FW |
| LU-10 | TAGTTCGAATCATGCATGATGTCTCTG | PDGFRA_RV |
| LU-25 | GATGCAGCTGCCTTATGACTCA | PDGFRA_FW |
| LU-25 | CAAGCTCAGATCTCTATTCTGCCAA | PDGFRA_RV |
| LU-26 | TGTCTGAACTGAAGATAATGACTCACCT | PDGFRA_FW |
| LU-26 | GATTTAAGCCTGATTGAACAGTTTTCACAA | PDGFRA_RV |
| LU-27 | GGAAAAATTGTGAAGATCTGTGACTTTGG | PDGFRA_FW |
| LU-27 | TCTAGAAGCAACACCTGACTTTAGAGATTA | PDGFRA_RV |
| LU-6 | ATTTTACAGAGTAACAGACTAGCTAGAGACA | PIK3CA_FW |
| LU-6 | AGAAACAGAGAATCTCCATTTTAGCACTTAC | PIK3CA_RV |
| LU-7 | ACAGCATGCCAATCTCTTCATAAATCT | PIK3CA_FW |
| LU-7 | CATGATGTGCATCATTCATTTGTTTCATG | PIK3CA_RV |
| LU-23 | CCTGAAGGTATTAACATCATTTGCTCCA | PIK3CA_FW |
| LU-23 | CCAGAGCCAAGCATCATTGAGAAA | PIK3CA_RV |
| LU-24 | TGAGCAAGAGGCTTTGGAGTATTT | PIK3CA_FW |
| LU-24 | AGAGTTATTAACAGTGCAGTGTGGAATC | PIK3CA_RV |

TABLE 2
5′ Phosphorylated Oligonucleotide
Sequences For The Colorectal Cancer Panel.
(SEQ ID NO: 67-SEQ ID NO: 134)

| Amplicon | | Gene |
|---|---|---|
| ID | Oligo Sequence 5′-3′ | Symbol |
| CRC-17 | ACTCCATCGAGATTTCACTGTAGCTA | BRAF_FW |
| CRC-17 | TCTCTTACCTAAACTCTTCATAATGCTTGC | BRAF_RV |
| CRC-18 | CATACTTACCATGCCACTTTCCCTT | BRAF_FW |
| CRC-18 | CTTTTTCTGTTTGGCTTGACTTGACTT | BRAF_RV |
| CRC-33 | AATGACTTTCTAGTAACTCAGCAGCAT | BRAF_FW |
| CRC-33 | CCTCACAGTAAAAATAGGTGATTTTGGTC | BRAF_RV |
| CRC-34 | CCTATTATGACTTGTCACAATGTCACCA | BRAF_FW |
| CRC-34 | TAGACGGGACTCGAGTGATGATT | BRAF_RV |
| CRC-6 | GTGGTCTCCCATACCCTCTCA | ERBB2_FW |
| CRC-6 | ACATGGTCTAAGAGGCAGCCATA | ERBB2_RV |
| CRC-23 | GCTGGTGACACAGCTTATGC | ERBB2_FW |
| CRC-23 | CTCCGGAGAGACCTGCAAAG | ERBB2_RV |
| CRC-12 | TCCTAGAGTAAGCCAGGGCTTT | KIT_FW |
| CRC-12 | CCTTACATTCAACCGTGCCATT | KIT_RV |
| CRC-13 | TCTGACCTACAAATATTTACAGGTAACCAT | KIT_FW |
| CRC-13 | CATTTATCTCCTCAACAACCTTCCACT | KIT_RV |
| CRC-14 | GCCATGACTGTCGCTGTAAAGA | KIT-FW |
| CRC-14 | GGTAACTCAGGACTTTGAGTTCAGAC | KIT_RV |
| CRC-15 | CACCTTCTTTCTAACCTTTTCTTATGTGC | KIT_FW |
| CRC-15 | CTTATAAAGTGCAGCTTCTGCATGATC | KIT_RV |
| CRC-16 | GGTTTTCTTTTCTCCTCCAACCTAATAGT | KIT_FW |
| CRC-16 | GTCAAGCAGAGAATGGGTACTCA | KIT_RV |
| CRC-29 | AGTTCTATAGATTCTAGTGCATTCAAGCAC | KIT_FW |
| CRC-29 | GATATGGTAGACAGAGCCTAAACATCC | KIT_RV |
| CRC-30 | CCCACAGAAACCCATGTATGAAGTAC | KIT_FW |
| CRC-30 | CCCAAAAAGGTGACATGGAAAGC | KIT_RV |
| CRC-31 | TTGACAGAACGGGAAGCCCTCAT | KIT_FW |
| CRC-31 | GTCATGTTTTGATAACCTGAGAGACAATAA | KIT_RV |
| CRC-32 | CGTGATTCATTTATTTGTTCAAAGCAGGAA | KIT_FW |
| CRC-32 | GCCTTGATTGCAAACCCTTATGAC | KIT_RV |
| CRC-4 | TGTCAGCTTATTATATTCAATTTAAACCCACCT | KRAS_FW |
| CRC-4 | CAGGTCAAGAGGAGTACAGTGC | KRAS_RV |
| CRC-5 | CAAAGAATGGTCCTGCACCAGTA | KRAS_FW |
| CRC-5 | AAGGCCTGCTGAAAATGACTGAATATA | KRAS_RV |
| CRC-21 | TCCTCATGTACTGGTCCCTCATT | KRAS_FW |
| CRC-21 | GGTGCACTGTAATAATCCAGACTGT | KRAS_RV |
| CRC-22 | GCTGTATCGTCAAGGCACTCTTG | KRAS_FW |
| CRC-22 | AGGTACTGGTGGAGTATTTGATAGTGTATT | KRAS_RV |
| CRC-1 | AACAACCTAAAACCAACTCTTCCCAT | KRAS_FW |
| CRC-1 | TGCCATCAATAATAGCAAGTCATTTGC | KRAS_RV |
| CRC-2 | TCTTGTCCAGCTGTATCCAGTATGT | KRAS_FW |
| CRC-2 | GATGCTTATTTAACCTTGGCAATAGCATT | KRAS_RV |
| CRC-3 | CTCCAACCACCACCAGTTTGTA | KRAS_FW |
| CRC-3 | GGAAGGTCACACTAGGGTTTTCAT | KRAS_RV |
| CRC-19 | GCTCCTAGTACCTGTAGAGGTTAATATCC | KRAS_FW |
| CRC-19 | GTTATAGATGGTGAAACCTGTTTGTTGG | KRAS_RV |
| CRC-20 | CGACAAGTGAGAGACAGGATCA | KRAS_FW |
| CRC-20 | TCTTGCTGGTGTGAAATGACTGAG | KRAS_RV |
| CRC-9 | GGTGCACTGGGACTTTGGTAAT | PDGFRA_FW |
| CRC-9 | TCCATCTCTTGGAAACTCCCATCT | PDGFRA_RV |
| CRC-10 | TCTGAGAACAGGAAGTTGGTAGCT | PDGFRA_FW |
| CRC-10 | CAGCAAGTTTACAATGTTCAAATGTGG | PDGFRA_RV |
| CRC-11 | GGGTGATGCTATTCAGCTAGAGA | PDGFRA_FW |
| CRC-11 | TAGTTCGAATCATGCATGATGTCTCTG | PDGFRA_RV |
| CRC-26 | GATGCAGCTGCCTTATGACTCA | PDGFRA_FW |
| CRC-26 | CAAGCTCAGATCTCTATTCTGCCAA | PDGFRA_RV |
| CRC-27 | TGTCTGAACTGAAGATAATGACTCACCT | PDGFRA_FW |
| CRC-27 | GATTTAAGCCTGATTGAACAGTTTTCACAA | PDGFRA_RV |
| CRC-28 | GGAAAAATTGTGAAGATCTGTGACTTTGG | PDGFRA_FW |
| CRC-28 | TCTAGAAGCAACACCTGACTTTAGAGATTA | PDGFRA_RV |
| CRC-7 | ATTTTACAGAGTAACAGACTAGCTAGAGACA | PIK3CA_FW |
| CRC-7 | AGAAACAGAGAATCTCCATTTTAGCACTTAC | PIK3CA_RV |
| CRC-8 | ACAGCATGCCAATCTCTTCATAAATCT | PIK3CA_FW |
| CRC-8 | CATGATGTGCATCATTCATTTGTTTCATG | PIK3CA_RV |
| CRC-24 | CCTGAAGGTATTAACATCATTTGCTCCA | PIK3CA_FW |
| CRC-24 | CCAGAGCCAAGCATCATTGAGAAA | PIK3CA_RV |
| CRC-25 | TGAGCAAGAGGCTTTGGAGTATTT | PIK3CA_FW |
| CRC-25 | AGAGTTATTAACAGTGCAGTGTGGAATC | PIK3CA_RV |

TABLE 3
5′ Phosphorylated Oligonucleotide
Sequences For The Pan-Cancer Panel.
(SEQ ID NO: 135-SEQ ID NO: 278)

| Amplicon | | Gene_ |
|---|---|---|
| ID | Oligo_Sequence_5′-3′ | Symbol |
| PAN-CA-35 | AGTATGCGCTGAAGCTCCATTT | ABL1_FW |
| PAN-CA-35 | CAGGTTAGGGTGTTTGATCTCTTTCA | ABL1_RV |
| PAN-CA-36 | CGTGTTGAAGTCCTCGTTGTCT | ABL1_FW |
| PAN-CA-36 | GAGATCTGAGTGGCCATGTACAG | ABL1_RV |
| PAN-CA-37 | GATCTCGTCAGCCATGGAGTAC | ABL1_FW |
| PAN-CA-37 | CCAGCACTGAGGTTAGAAGCTG | ABL1_RV |
| PAN-CA-70 | AGTTCTTGAAAGAAGCTGCAGTCA | ABL1_FW |
| PAN-CA-70 | TATTCCAACGAGGTTTTGTGCAGT | ABL1_RV |
| PAN-CA-71 | CCCGTTCTATATCATCACTGAGTTCA | ABL1_FW |
| PAN-CA-71 | CCTGTGGATGAAGTTTTTCTTCTCCA | ABL1_RV |
| PAN-CA-72 | CAGAAGATTCGCAGAAGCTCATCT | ABL1_FW |
| PAN-CA-72 | AATCAGAGGCCTGAAACCAATCTAAAT | ABL1_RV |
| PAN-CA-18 | GGGTGAGGCAGTCTTTACTCAC | ALK_FW |
| PAN-CA-18 | GCCGTTGTACACTCATCTTCCTAG | ALK_RV |
| PAN-CA-19 | GGGTGTCTCTCTGTGGCTTTAC | ALK_FW |
| PAN-CA-19 | CTCTGTAGGCTGCAGTTCTCAG | ALK_RV |
| PAN-CA-54 | TTGGCACAACAACTGCAGCAAA | ALK_FW |
| PAN-CA-54 | AGCAAATTCAACCACCAGAACATTG | ALK_RV |
| PAN-CA-33 | AATGACTTTCTAGTAACTCAGCAGCAT | BRAF_FW |
| PAN-CA-33 | CCTCACAGTAAAAATAGGTGATTTTGGTC | BRAF_RV |
| PAN-CA-34 | CCTATTATGACTTGTCACAATGTCACCA | BRAF_FW |
| PAN-CA-34 | TAGACGGGACTCGAGTGATGATT | BRAF_RV |
| PAN-CA-68 | ACTCCATCGAGATTTCACTGTAGCTA | BRAF_FW |
| PAN-CA-68 | TCTCTTACCTAAACTCTTCATAATGCTTGC | BRAF_RV |
| PAN-CA-69 | CATACTTACCATGCCACTTTCCCTT | BRAF_FW |
| PAN-CA-69 | CTTTTTCTGTTTGGCTTGACTTGACTT | BRAF_RV |
| PAN-CA-2 | GAAATTTAACAGGGTGTTGTTGTGCA | DDR2_FW |
| PAN-CA-2 | CTGTTCATCTGACAGCTGGGAATA | DDR2_RV |
| PAN-CA-9 | TGTTTGTTTGTTTAACTTTGTGTCGCTA | DNMT3A_FW |
| PAN-CA-9 | CACTATACTGACGTCTCCAACATGAG | DNMT3A_RV |
| PAN-CA-10 | CCAGGACGTTTGTGGAAAACAAG | DNMT3A_FW |
| PAN-CA-10 | ATGAATGAGAAAGAGGACATCTTATGGTG | DNMT3A_RV |
| PAN-CA-11 | CCCAGCAGAGGTTCTAGACG | DNMT3A_FW |
| PAN-CA-11 | GCTGTTATCCAGGTTTCTGTTGTT | DNMT3A_RV |
| PAN-CA-12 | CAGGAGCTTTCACCAACCTGT | DNMT3A_FW |
| PAN-CA-12 | CGCTGTTTCATGCTCCTCCTT | DNMT3A_RV |
| PAN-CA-13 | GAGATGTCCCTCTTGTCACTAACG | DNMT3A_FW |
| PAN-CA-13 | CCAGCTGATGGCTTTCTCTTCC | DNMT3A_RV |
| PAN-CA-14 | CCCAATCACCAGATCGAATGG | DNMT3A_FW |
| PAN-CA-14 | CCTCTCTTTCGTGTCAAAGGACTTC | DNMT3A_RV |
| PAN-CA-15 | CCCACAGCATGGACATACATG | DNMT3A_FW |
| PAN-CA-15 | GAAGGACTTGGGCATTCAGGT | DNMT3A_RV |
| PAN-CA-16 | CTAACCATCATTTCGTTTTGCCAGA | DNMT3A_FW |
| PAN-CA-16 | TCCAAAGGTTTACCCACCTGTC | DNMT3A_RV |
| PAN-CA-17 | CTCAGCCAAGGGAGCTCGAGA | DNMT3A_FW |
| PAN-CA-17 | CTGGAACTGCTACATGTGCG | DNMT3A_RV |
| PAN-CA-45 | GATGACTGGCACGCTCCAT | DNMT3A_FW |
| PAN-CA-45 | GCTGTGTGGTTAGACGGCTTC | DNMT3A_RV |
| PAN-CA-46 | CCGGGTACCTTTCCATTTCAGTG | DNMT3A_FW |
| PAN-CA-46 | GCTTATTCCTCTTTTCTCCTCTTCATCTAG | DNMT3A_RV |
| PAN-CA-47 | GGAAAAGGAAATAAGGAACATGGCAGA | DNMT3A_FW |
| PAN-CA-47 | GGGTAACCTTCCCGGTATGA | DNMT3A_RV |
| PAN-CA-48 | CAGCTCCACAATGCAGATGAGA | DNMT3A_FW |
| PAN-CA-48 | CTTCTGGCTCTTTGAGAATGTGGT | DNMT3A_RV |
| PAN-CA-49 | GAGGAAGCCTATGTGCGGAA | DNMT3A_FW |
| PAN-CA-49 | CGTTGCCTTTATCCTCCCAGAT | DNMT3A_RV |
| PAN-CA-50 | GGACAAATGGAAGATAAGGAGAAAAAGAGG | DNMT3A_FW |
| PAN-CA-50 | GTCCGCAGCGTCACACAGAAG | DNMT3A_RV |
| PAN-CA-51 | CCGAGGCAATGTAGCGGTC | DNMT3A_FW |
| PAN-CA-51 | CTTGGGCCTACAGCTGACC | DNMT3A_RV |
| PAN-CA-52 | GATGGGCTTCCTCTTCTCAGC | DNMT3A_FW |
| PAN-CA-52 | AGGGTGTGTGGGTCTAGGAG | DNMT3A_RV |
| PAN-CA-53 | CCAGCACTCACAAATTCCTGGT | DNMT3A_FW |
| PAN-CA-53 | CCAGGCAGCCATTAAGGAAGAC | DNMT3A_RV |
| PAN-CA-29 | GGTGACCCTTGTCTCTGTGTTC | EGFR_FW |
| PAN-CA-29 | AGGGACCTTACCTTATACACCGT | EGFR_RV |
| PAN-CA-30 | CTGGTAACATCCACCCAGATCA | EGFR_FW |
| PAN-CA-30 | GGAGATGTTGCTTCTCTTAATTCCTTG | EGFR_RV |
| PAN-CA-31 | CATGCGAAGCCACACTGAC | EGFR_FW |
| PAN-CA-31 | GTTCCCGGACATAGTCCAGG | EGFR_RV |
| PAN-CA-64 | GGAAACTGAATTCAAAAAGATCAAAGTGCT | EGFR_FW |
| PAN-CA-64 | GGAAATATACAGCTTGCAAGGACTCT | EGFR_RV |
| PAN-CA-65 | TGAGAAAGTTAAAATTCCCGTCGCTAT | EGFR_FW |
| PAN-CA-65 | CTGCCAGACATGAGAAAAGGTG | EGFR_RV |
| PAN-CA-66 | TGTTTCAGGGCATGAACTACTTGG | EGFR_FW |
| PAN-CA-66 | ACCTCCTTACTTTGCCTCCTTCT | EGFR_RV |
| PAN-CA-44 | GTGGTCTCCCATACCCTCTCA | ERBB2_FW |
| PAN-CA-44 | AGCCATAGGGCATAAGCTGTG | ERBB2_RV |
| PAN-CA-6 | ATGTTACCATAAATCAAAAATGCACCACA | FLT3_FW |
| PAN-CA-6 | ACTTTGGATTGGCTCGAGATATCATG | FLT3_RV |
| PAN-CA-7 | ATCTTTGTTGCTGTCCTTCCACT | FLT3_FW |
| PAN-CA-7 | ATCTTTAAAATGCACGTACTCACCATTTG | FLT3_RV |
| PAN-CA-8 | TTGGAAACTCCCATTTGAGATCATATTCAT | FLT3_FW |
| PAN-CA-8 | GCCTATTCCTAACTGACTCATCATTTCA | FLT3_RV |
| PAN-CA-42 | CCCTGACAACATAGTTGGAATCACT | FLT3_FW |
| PAN-CA-42 | CACAGTAAATAACACTCTGGTGTCATTCT | FLT3_RV |
| PAN-CA-43 | AGACAAATGGTGAGTACGTGCAT | FLT3_FW |
| PAN-CA-43 | TCCTCAGATAATGAGTACTTCTACGTTGAT | FLT3_RV |
| PAN-CA-24 | GAGTTCTATAGATTCTAGTGCATTCAAGCA | KIT_FW |
| PAN-CA-24 | GATATGGTAGACAGAGCCTAAACATCC | KIT_RV |
| PAN-CA-25 | CCCACAGAAACCCATGTATGAAGTAC | KIT_FW |
| PAN-CA-25 | CCCAAAAAGGTGACATGGAAAGC | KIT_RV |
| PAN-CA-26 | TTGACAGAACGGGAAGCCCTCAT | KIT_FW |
| PAN-CA-26 | GTCATGTTTTGATAACCTGACAGACAATAA | KIT_RV |
| PAN-CA-27 | CGTGATTCATTTATTTGTTCAAAGCAGGAA | KIT_FW |
| PAN-CA-27 | GCCTTGATTGCAAACCCTTATGAC | KIT_RV |
| PAN-CA-59 | TCTGACCTACAAATATTTACAGGTAACCAT | KIT_FW |
| PAN-CA-59 | CATTTATCTCCTCAACAACCTTCCACT | KIT_RV |
| PAN-CA-60 | GCCATGACTGTCGCTGTAAAGA | KIT_FW |
| PAN-CA-60 | GGTAACTCAGGACTTTGAGTTCAGAC | KIT_RV |
| PAN-CA-61 | CACCTTCTTTCTAACCTTTTCTTATGTGC | KIT_FW |
| PAN-CA-61 | CTTATAAAGTGCAGCTTCTGCATGATC | KIT_RV |
| PAN-CA-62 | GGTTTTCTTTTCTCCTCCAACCTAATAGT | KIT_FW |
| PAN-CA-62 | GTCAAGCAGAGAATGGGTACTCA | KIT_RV |
| PAN-CA-4 | TCCTCATGTACTGGTCCCTCAT | KRAS_FW |
| PAN-CA-4 | GGTGCACTGTAATAATCCAGACTGT | KRAS_RV |
| PAN-CA-5 | GCTGTATCGTCAAGGCACTCTTG | KRAS_FW |
| PAN-CA-5 | AGGTACTGGTGGAGTATTTGATAGTGTATT | KRAS_RV |
| PAN-CA-41 | CAAAGAATGGTCCTGCACCAGTA | KRAS_FW |
| PAN-CA-41 | AAGGCCTGCTGAAAATGACTGAATATA | KRAS_RV |
| PAN-CA-28 | GAATTTTCTAAAGGTATCTCTCTCGGTGTA | NPM1_FW |
| PAN-CA-28 | CCAGTTACCTCTTGGTCAGTCATC | NPM1_RV |
| PAN-CA-63 | CTTAATAGGGTGGTTCTCTTCCCAAAG | NPM1_FW |
| PAN-CA-63 | ACACTTAAAAAGGGTAAAGGCAGAATCATA | NPM1_RV |
| PAN-CA-1 | ATCCGCAAATGACTTGCTATTATTGATG | NRAS_FW |
| PAN-CA-1 | CCCAGGATTCTTACAGAAAACAAGTG | NRAS_RV |
| PAN-CA-39 | CCTCACCTCTATGGTGGGATCA | NRAS_FW |
| PAN-CA-39 | CGCCAATTAACCCTGATTACTGGT | NRAS_RV |
| PAN-CA-21 | GGTGCACTGGGACTTTGGTAAT | PDGFRA_FW |
| PAN-CA-21 | TCCATCTCTTGGAAACTCCCATCT | PDGFRA_RV |
| PAN-CA-22 | TCTGAGAACAGGAAGTTGGTAGCT | PDGFRA_FW |
| PAN-CA-22 | CAGCAAGTTTACAATGTTCAAATGTGG | PDGFRA_RV |
| PAN-CA-23 | GGGTGATGCTATTCAGCTACAGA | PDGFRA_FW |
| PAN-CA-23 | TAGTTCGAATCATGCATGATGTCTCTG | PDGFRA_RV |
| PAN-CA-56 | GATGCAGCTGCCTTATGACTCA | PDGFRA_FW |
| PAN-CA-56 | CAAGCTCAGATCTCTATTCTGCCAA | PDGFRA_RV |
| PAN-CA-57 | TGTCTGAACTGAAGATAATGACTCACCT | PDGFRA_FW |
| PAN-CA-57 | GATTTAAGCCTGATTGAACAGTTTTCACAA | PDGFRA_RV |
| PAN-CA-58 | GGAAAAATTGTGAAGATCTGTGACTTTGG | PDGFRA_FW |
| PAN-CA-58 | TCTAGAAGCAACACCTGACTTTAGAGATTA | PDGFRA_RV |
| PAN-CA-20 | TAAGGGAAAATGACAAAGAACAGCTCA | PIK3CA_FW |
| PAN-CA-20 | GCTGAGATCAGCCAAATTCAGTTATTTTT | PIK3CA_RV |
| PAN-CA-55 | CTTTTGATGACATTGCATACATTCGAAAGA | PIK3CA_FW |
| PAN-CA-55 | CAGTTATCTTTTCAGTTCAATGCATGCT | PIK3CA_RV |
| PAN-CA-3 | TACGCAGCCTGTACCCAGTG | RET_FW |
| PAN-CA-3 | TTGTGGTAGCAGTGGATGCA | RET_RV |
| PAN-CA-40 | CCCTCCTTCCTAGAGAGTTAGAGT | RET_FW |
| PAN-CA-40 | CAAGAGAGCAACACCCACACTTA | RET_RV |
| PAN-CA-32 | CCCAGCTGGGTGAACTTTGAG | SMO_FW |
| PAN-CA-32 | CAGCTGAAGGTAATGAGCACAAAG | SMO_RV |
| PAN-CA-67 | CATTTTTGGCTTCCTGGCCTTT | SMO_FW |
| PAN-CA-67 | GGTGGGTGTCTTTATGGCCTT | SMO_RV |
| PAN-CA-38 | CCAGTCCCTTACTTGTTCAGCT | TSC1_FW |
| PAN-CA-38 | TGCCAAAGACAGCCCATCATTT | TSC1_RV |

Conclusion:

A new robust targeted-NGS method has been developed in order to provide clinicians and researchers with key mutation data from patients' specimens as soon as possible. This can help such clinicians and researchers to decide which therapeutic options (personalized medicine) or biological applications are optimal to treat the patients with specific mutations. For example, this application can be the screening of lung cancer specimens to detect tumor driven and drug-sensitive mutations in the EGFR gene, which can benefit patients from the tyrosine kinase inhibitors (TKI, i.e. Gefitinib or Erlotinib) treatment. By combining with the DNA extraction kit and computing system for mutant analysis described herein, the amplicon preparation method will be able to provide the key mutation data to patients, medical doctors, and researchers within 36 hours (next day).

### Example 3

### Database-Associated Non-Preprocessing Analysis (DanPA) of Next-Generation Sequencing (NGS)

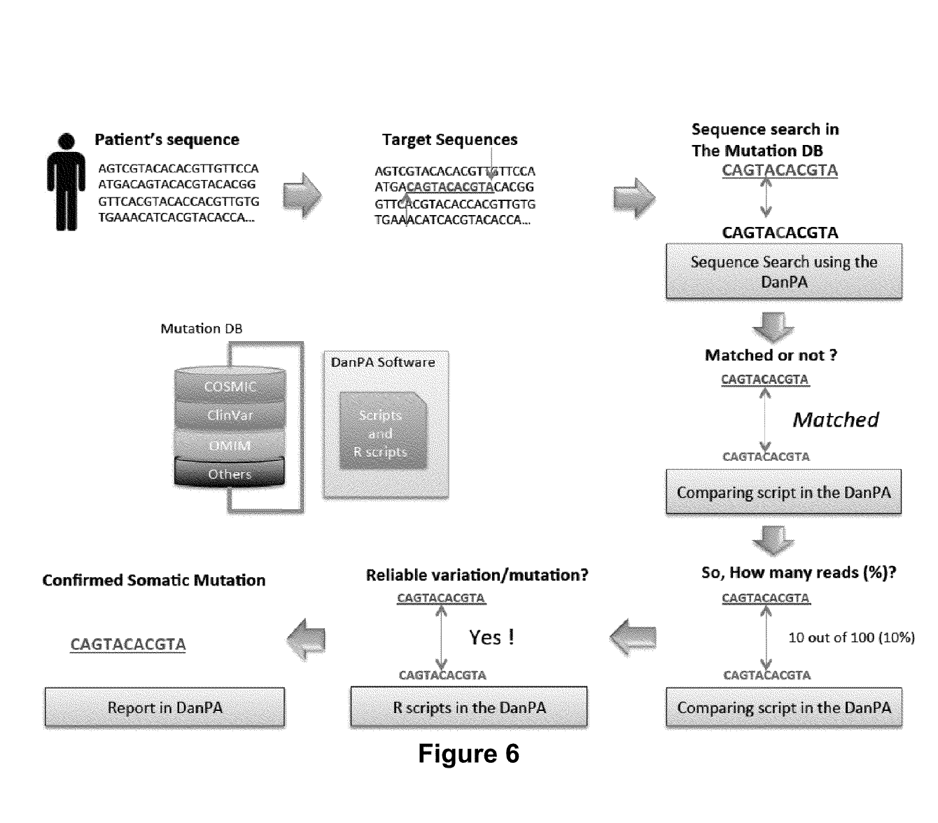

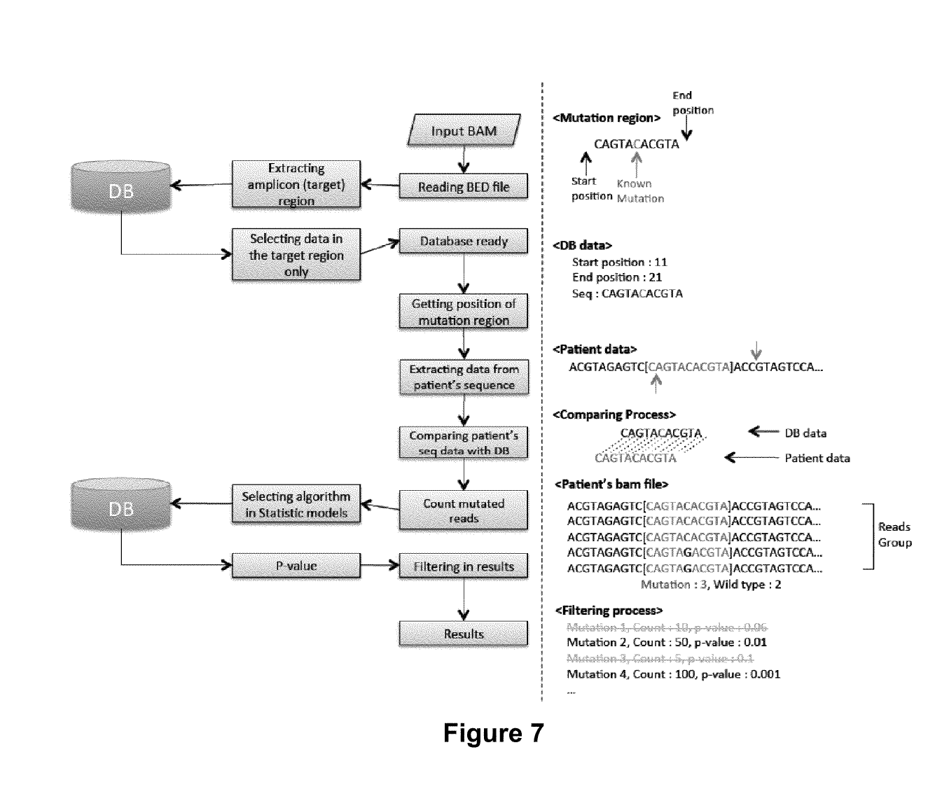

Methodology/Principal Findings: A new data analysis tool called DanPA that provides fast, accurate, and robust NGS data analysis. DanPA was developed mainly for targeted sequencing analysis, though it can also be used for the whole exome or genome sequencing data analysis. The DanPA detects any kind of reported mutations registered in the database such as Catalogue Of Somatic Mutations In Cancer (COSMIC), the biggest and robust cancer mutation database (FIGS. 6 and 7). There are more than 1.5 million registered mutations in the COSMIC, and any additional database can be connected to the DanPA for the mutation screening (FIG. 6). Thus, it is assumed that any genetic variations or mutations not registered in these databases would be non-pathogenic or extremely rare mutations with a very limited clinical or biological effect. If necessary, additional or new mutations (probably after its biological and clinical role in certain diseases are proven) can be easily added to the mutation databases.

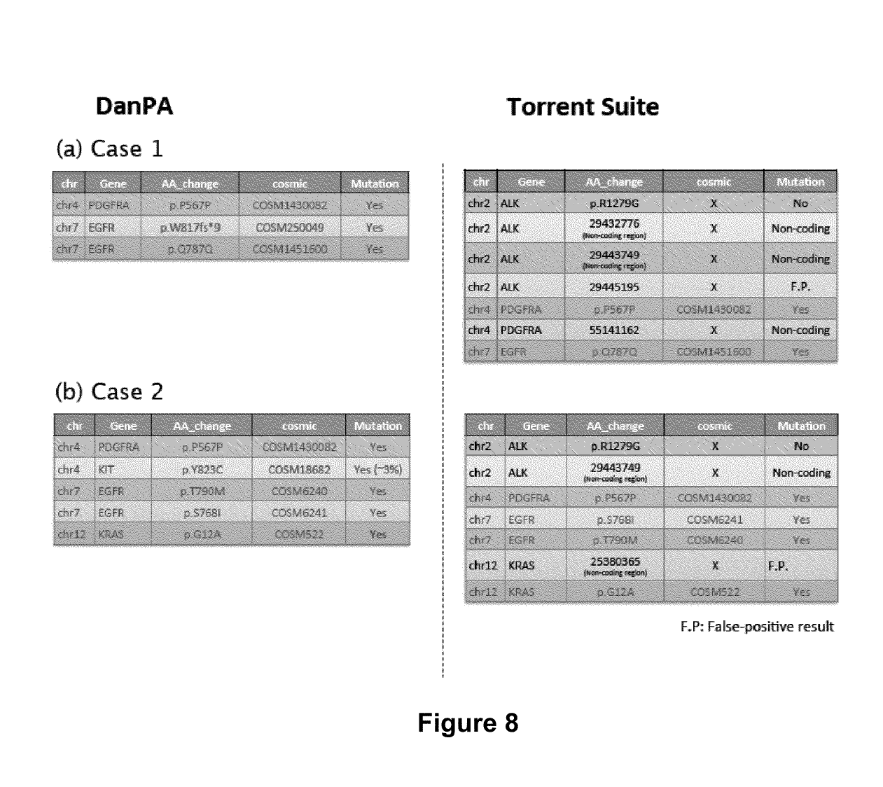

A classical NGS data analysis procedure comprises of several steps (unmapped sequence re-alignment, de-duplication, indel realignment, and base quality score recalibration) called ‘pre-processing’ of the NGS data analysis. There are several NGS data analysis tools (i.e. SAMtools, GATK, Picard, and Torrent Suite/Reporter) mainly developed for the large scale of the NGS data analysis. Although these programs use different algorithms for each of the preprocessing steps, they generally work according to the following steps: unmapped sequence realignment, de-duplication, indel realignment, and base quality score recalibration. DanPA skips these pre-processing steps and connects the designated database for detecting mutations. Thus, any kind of registered mutations can be robustly detected by DanPA. The best example is exon 19 deletions of the EGFR gene. Correct mutation information of this gene is important and fundamental for the clinical decision in cancer patients. Lung cancer patients with EGFR mutations such as exon 19 deletions or L858R mutation are responsive to the tyrosine kinase inhibitor (TKI), Gefitinib or Erlotinib. However, exon 19 deletions tend to be more than 15 bp deletion or an even combination of both deletion and insertion (indel) which is very hard to be detected by other NGS analysis program. Moreover, the Ion Torrent system, one of two leading commercial sequencing platforms, has a serious problem with detecting (complicating) insertions and deletions like EGFR exon 19 mutations. In the application of DanPA to the Ion Torrent data, however, there was no problem detecting these kinds of complicated mutations as long as they were registered in the database. The comparison data using the DanPA and the Torrent Suite (official data analyses program supported by the Ion Torrent) are shown in FIG. 8. Another one of DanPA's big advantages in detecting mutation is a dramatic reduction of a false-positive call or sequencing error as it selects only database-registered mutations. It has been known that NGS has a high false positive rate in a homopolymer region. As DanPA detects mutations by directly connecting database with a designated cut-off level (allele frequency: i.e. 3% of mutant allele frequency), most of those false-positive mutation calls are removed and only clear somatic mutations are detected.

Tables 4 and 5 summarize another experiment utilizing the subject ‘NextDay Seq’ direct amplification and ligation amplicon sample library preparation followed by next generation sequencing and data analysis using DanPA as described herein. Table 4 provides a summary of the clinical and biological samples used in the experiment and Table 5 provides a summary of the mutations uncovered from the 866 FFPE samples used in the experiment.

TABLE 4

| Sample type | Number of samples |
|---|---|
| FFPE | 866 |
| Fresh-frozen tissues | 431 |
| Plasmid | 114 |
| Cell lines | 18 |
| Others | 401 |

TABLE 5

| | | | Number of |
|---|---|---|---|
| Gene | Nucleotide Change | Amino Acid Change | samples |
| PDGFRA | c.1701A > G | p.P567P | 815 |
| EGFR | c.2361G > A | p.Q787Q | 296 |
| EGFR | c.2573T > G | p.L858R | 150 |
| EGFR | c.2235_2249del15 | p.E746_A750delELREA | 61 |
| NRAS | c.38G > A | p.G13D | 56 |
| EGFR | c.2369C > T | p.T790M | 50 |
| KRAS | c.35G > A | p.G12D | 48 |
| BRAF | c.1799T > A | p.V600E | 41 |
| PIK3CA | c.1633G > A | p.E545K | 40 |
| EGFR | c.2156G > C | p.G719A | 39 |
| PDGFRA | c.2472C > T | p.V824V | 35 |
| PIK3CA | c.3140A > G | p.H1047R | 28 |
| EGFR | c.2236_2250del15 | p.E746_A750delELREA | 27 |
| EGFR | c.2303G > T | p.S768I | 27 |
| KRAS | c.35G > T | p.G12V | 23 |
| EGFR | c.2582T > A | p.L861Q | 22 |
| EGFR | c.2155G > A | p.G719S | 20 |
| EGFR | c.2240_2257del18 | p.L747_P753 > S | 19 |
| EGFR | c.2238_2252del15 | p.L747_T751delLREAT | 15 |
| KRAS | c.183A > C | p.Q61H | 14 |
| EGFR | c.2239_2248TTAAGAGAAG > C | p.L747_A750 > P | 14 |
| PIK3CA | c.1645G > A | p.D549N | 11 |
| KRAS | c.34G > T | p.G12C | 11 |
| EGFR | c.2237_2255 > T | p.E746_S752 > V | 9 |
| PIK3CA | c.3075C > T | p.T1025T | 8 |
| PIK3CA | c.3140A > T | p.H1047L | 8 |
| EGFR | c.2126A > C | p.E709A | 8 |
| EGFR | c.2155G > T | p.G719C | 7 |
| PIK3CA | c.1624G > A | p.E542K | 7 |
| EGFR | c.2579A > T | p.K860I | 6 |
| KRAS | c.182A > T | p.Q61L | 6 |
| KRAS | c.182A > G | p.Q61R | 6 |
| EGFR | c.2311_2312insGCGTGGACA | p.D770_N771insSVD | 5 |
| EGFR | c.2307_2308insGCCAGCGTG | p.V769_D770insASV | 5 |
| KRAS | c.35G > C | p.G12A | 5 |
| NRAS | c.38G > T | p.G13V | 5 |
| KRAS | c.183A > T | p.Q61H | 4 |
| EGFR | c.2310_2311insGGGGAC | p.D770_N771insGD | 4 |
| BRAF | c.1801A > G | p.K601E | 4 |
| EGFR | c.2316_2317ins9 | p.P772_H773insDNP | 4 |
| KRAS | c.181C > A | p.Q61K | 4 |
| EGFR | c.2125G > A | p.E709K | 4 |
| PIK3CA | c.1635G > T | p.E545D | 4 |
| EGFR | c.2175G > A | p.T725T | 3 |
| EGFR | c.2065G > A | p.V689M | 3 |
| KRAS | c.37G > T | p.G13C | 3 |
| DNMT3A | c.2222C > T | p.A741V | 3 |
| ERBB2 | c.2379G > A | p.T793T | 3 |
| KRAS | c.34G > A | p.G12S | 3 |
| EGFR | c.2457G > A | p.V819V | 3 |
| EGFR | c.2239_2256del18 | p.L747_S752delLREATS | 2 |
| EGFR | c.2573_2574TG > GT | p.L858R | 2 |
| BRAF | c.1790T > G | p.L597R | 2 |
| EGFR | c.2276T > C | p.I759T | 2 |
| EGFR | c.2240T > C | p.L747S | 2 |
| EGFR | c.2236_2259 > ATCTCG | p.E746_P753 > IS | 2 |
| EGFR | c.2254_2277de124 | p.S752_I759delSPKANKEI | 2 |
| EGFR | c.2497T > G | p.L833V | 2 |
| PIK3CA | c.3073A > T | p.T1025S | 2 |
| EGFR | c.2238_2248 > GC | p.L747_A750 > P | 2 |
| DNMT3A | c.2645G > A | p.R882H | 2 |
| PIK3CA | c.3172A > G | p.I1058V | 2 |
| EGFR | c.2126A > G | p.E709G | 2 |
| EGFR | c.2253_2276del24 | p.S752_I759delSPKANKEI | 2 |
| PIK3CA | c.3139C > T | p.H1047Y | 2 |
| EGFR | c.2318_2319insCCCCCA | p.H773_V774insPH | 1 |
| EGFR | c.2360A > G | p.Q787R | 1 |
| BRAF | c.1797_1798insACA | p.T599_V600insT | 1 |
| EGFR | c.2572_2573CT > AG | p.L858R | 1 |
| PIK3CA | c.3132T > A | p.N1044K | 1 |
| EGFR | c.2580A > G | p.K860K | 1 |
| EGFR | c.2239_2256 > CAA | p.L747_S752 > Q | 1 |
| EGFR | c.2063T > C | p.L688P | 1 |
| BRAF | c.1807C > T | p.R603* | 1 |
| EGFR | c.2236_2251del17 | p.E746_T751fs | 1 |
| KIT | c.1673_1674insTCC | p.K558 > NP | 1 |
| ALK | c.3645G > A | p.P1215P | 1 |
| PIK3CA | c.3184A > G | p.I1062V | 1 |
| EGFR | c.2494C > T | p.R832C | 1 |
| EGFR | c.2092G > A | p.A698T | 1 |
| EGFR | c.2492G > A | p.R831H | 1 |
| EGFR | c.2239_2240TT > CC | p.L747P | 1 |
| KRAS | c.57G > T | p.L19F | 1 |
| PIK3CA | c.3118A > G | p.M1040V | 1 |
| EGFR | c.2414A > G | p.H805R | 1 |
| EGFR | c.2491C > T | p.R831C | 1 |
| EGFR | c.? | p.D771_? | 1 |
| PIK3CA | c.1637A > G | p.Q546R | 1 |
| EGFR | c.2348C > T | p.T783I | 1 |
| EGFR | c.2393T > A | p.L798H | 1 |
| EGFR | c.2180A > G | p.Y727C | 1 |
| EGFR | c.2537A > G | p.K846R | 1 |
| EGFR | c.2319_2320insAACCCCCAC | p.H773_V774insNPH | 1 |
| EGFR | c.2237_2257 > TCT | p.E746_P753 > VS | 1 |
| KIT | c.2466T > G | p.N822K | 1 |
| EGFR | c.2274A > G | p.E758E | 1 |
| DNMT3A | c.2644C > T | p.R882C | 1 |
| KIT | c.1486G > A | p.D496N | 1 |
| ALK | c.3830T > C | p.I1277T | 1 |
| PIK3CA | c.3129G > A | p.M1043I | 1 |
| KRAS | c.39C > T | p.G13G | 1 |
| KIT | c.1671G > A | p.W557* | 1 |
| EGFR | c.2239_2251 > C | p.L747_T751 > P | 1 |
| BRAF | c.1406G > C | p.G469A | 1 |
| ALK | c.3631A > G | p.T1211A | 1 |
| PDGFRA | c.2552C > T | p.S851L | 1 |
| EGFR | c.2311A > GGTT | p.N771 > GY | 1 |
| PIK3CA | c.1634A > C | p.E545A | 1 |
| EGFR | c.2237_2251 > TTC | p.E746_T751 > VP | 1 |
| EGFR | c.2410G > A | p.E804K | 1 |
| EGFR | c.2235_2252 > AAT | p.E746_T751 > I | 1 |
| ALK | c.3746A > G | p.D1249G | 1 |
| PIK3CA | c.3151T > C | p.W1051R | 1 |
| EGFR | c.2441T > C | p.L814P | 1 |
| EGFR | c.2512C > G | p.L838V | 1 |
| EGFR | Deletion | p.? | 1 |
| PIK3CA | c.1634A > G | p.E545G | 1 |
| KIT | c.1687_1716del30 | p.I563_D572del | 1 |
| EGFR | c.2281G > A | p.D761N | 1 |
| EGFR | c.2596G > A | p.E866K | 1 |
| EGFR | c.2296A > G | p.M766V | 1 |
| EGFR | c.2239_2248 > CCG | p.L747_E749 > P | 1 |
| PIK3CA | c.3148G > A | p.G1050S | 1 |
| EGFR | c.2232C > G | p.I744M | 1 |
| EGFR | c.2125G > C | p.E709Q | 1 |
| DNMT3A | c.1904G > A | p.R635Q | 1 |
| PIK3CA | c.1675_1680GTTGTT > A | p.V559_V560del | 1 |
| EGFR | c.2240_2251del12 | p.L747_T751 > S | 1 |
| EGFR | c.2239_2253 > GCT | p.L747_T751 > A | 1 |
| ALK | c.3635G > A | p.R1212H | 1 |
| EGFR | c.2239_2264 > GCCAA | p.L747_A755 > AN | 1 |
| PIK3CA | c.3127A > G | p.M1043V | 1 |
| EGFR | c.2392C > T | p.L798F | 1 |
| ALK | c.3509T > C | p.I1170T | 1 |
| EGFR | c.2310_2311insGGGTTT | p.D770_N771insGF | 1 |
| EGFR | c.2240_2254del15 | p.L747_T751delLREAT | 1 |
| ERBB2 | c.2329G > A | p.V777M | 1 |
| EGFR | c.2252_2276 > G | p.T751_I759 > S | 1 |
| EGFR | c.2527G > A | p.V843I | 1 |
| PIK3CA | c.3185T > C | p.I1062T | 1 |
| EGFR | c.2091A > G | p.E697E | 1 |
| EGFR | c.2375T > C | p.L792P | 1 |
| EGFR | c.2308-2309insAACCCC | p.N771_P772fs | 1 |

### CONCLUSION

A new NGS data analysis program, DanPA, was developed that directly connected to mutation databases. This tool can process the mutation analysis from the NGS data within one hour while other programs take easily more than one day. A fast data analysis is available because of skipping almost all pre-processing steps routinely used in other NGS analysis programs. The accuracy of the DanPA is also the best among the programs tested (GATK, Torrent Suite and Reporter, and SAMtools). Additionally, DanPA solves two problems associated with NGS applications (especially in the Ion Torrent sequencers): false negatives (i.e. indels and long-bp deletions of the EGFR gene) and false-positives (i.e. deletion or insertion in homopolymer regions). This fastest, simplest, and most accurate NGS analysis program will help clinicians and researchers identify meaningful clinical markers and genetic mechanisms in human diseases or any life science fields.

We claim:

1. A method for extracting nucleic acid from a preserved tissue sample, the method comprising the steps of:
(a) incubating the preserved tissue sample with a tissue digestion solution to form a tissue digestion mixture, wherein the tissue digestion solution is selected from the group consisting of:
(i) a tissue digestion solution comprising NaCl at a concentration of 10 mM to 140 mM, Na₂HPO₄ at a concentration of 0.5 mM to 10 mM, KH₂PO₄ at a concentration of 0.1 mM to 5 mM, and Tween 20;
(ii) a tissue digestion solution comprising NaCl at a concentration of 10 mM to 140 mM, Na₂HPO₄ at a concentration of 0.5 mM to 10 mM, KH₂PO₄ at a concentration of 0.1 mM to 5 mM, and Triton-X100;
(iii) a tissue digestion solution comprising NaCl at a concentration of 10 mM to 140 mM, Na₂HPO₄ at a concentration of 0.5 mM to 10 mM, and KH₂PO₄ at a concentration of 0.1 mM to 5 mM;
(iv) a tissue digestion solution comprising TAPS sodium salt at a concentration of 0.5 mM to 25 mM, DTT at a concentration of 0.05 mM to 5 mM, and KCl at a concentration of 0.2 mM to 200 mM;
(v) a tissue digestion solution comprising HEPES buffer at a concentration of 1 mM to 100 mM;
(vi) a tissue digestion solution comprising HEPES buffer at a concentration of 1 mM to 100 mM and Triton-X100;
(vii) a tissue digestion solution comprising HEPES buffer at a concentration of 1 mM to 100 mM and Tween 20;
(viii) a tissue digestion solution comprising TAPS sodium salt at a concentration of 0.5 mM to 25 mM, DTT at a concentration of 0.05 mM to 5 mM, KCl at a concentration of 0.2 mM to 200 mM, and Triton-X100;
(ix) a tissue digestion solution comprising a TAPS sodium salt at a concentration of 0.5 mM to 25 mM, DTT at a concentration of 0.05 mM to 5 mM, KCl at a concentration of 0.2 mM to 200 mM, and Tween 20; and
(x) a tissue digestion solution comprising a TAPS sodium salt at a concentration of 0.5 mM to 25 mM, KCl at a concentration of 0.2 mM to 200 mM, β-Mercaptoethanol at a concentration of 0.1 mM to 1 mM, and Triton-X100,
(b) heating the tissue digestion mixture at 80 to 110° C. for 1-30 minutes;
(c) adding a protease solution comprising a proteinase to the tissue digestion mixture to form a protein degradation mixture and incubating the protein degradation mixture at 50 to 70° C. for 1-30 minutes; and
(d) incubating the protein degradation mixture at 80 to 110° C. for 1-30 minutes; thereby extracting nucleic acid from the preserved tissue sample.

2. The method of claim 1, wherein the protease solution is selected from the group consisting of:
(a) a protease solution comprising Proteinase K at a concentration of 5 mg/ml to 60 mg/ml, Tris-HCl (pH 8.0) at a concentration of 1 mM to 50 mM and EDTA at a concentraiton of 0.1 to 10 mM;
(b) a protease solution comprising Proteinase K at a concentration of 5 mg/ml to 60 mg/ml and Tris-HCl (pH 8.0) at a concentration of 1 mM to 50 mM;
(c) a protease solution comprising Proteinase K at a concentration of 5 mg/ml to 60 mg/ml and EDTA at a concentration of 0.1 mM to 10 mM
(d) a protease solution comprising Proteinase K at a concentration of 5 mg/ml to 60 mg/ml; and
(e) a protease solution comprising Proteinase K at a concentration of 5 mg/ml to 60 mg/ml, Tris-HCl (pH 8.0) at a concentration of 0.2 mM to 50 mM, CaCl2 at a concentration of 0.1 mM to 10 mM and glycerol at a concentration of 20% to 70%.

3. The method of claim 1, wherein the heating (b) is at 99° C. for 5 minutes.

4. The method of claim 1, wherein the incubating the protein degradation mixture (c) is at 60° C. for 5 minutes.

5. The method of claim 1, wherein the incubating the protein degradation mixture (d) is at 99° C. for 5 minutes.

6. A method for making a targeted nucleic acid amplicon library from a tissue sample, the method comprising the steps of:
(a) amplifying nucleic acid extracted from a tissue sample, the step of amplification using 5′ phosphorylated oligonucleotides that target a nucleic acid of interest; and
(b) directly ligating an oligonucleotide comprising an adaptor nucleic acid and a bar code nucleic acid to each of the amplified target nucleic acids, thereby making a targeted nucleic acid amplicon library.

7. The method of claim 6, further comprising the step of purifying the amplified target nucleic acid of (a) prior to directly ligating an oligonucleotide (b).

8. A method of detecting a mutation in a tissue sample target nucleic acid sequence without preprocessing of sequence data, the method comprising the steps of:
(a) obtaining a tissue sample target nucleic acid sequence data and database target nucleic acid sequence data, wherein the database target nucleic acid sequence data is located in a mutation database;
(b) comparing the tissue sample target nucleic acid sequence data with the database target nucleic acid sequence data to determine if the sample target nucleic acid sequence data contains a registered mutation from the mutation database;
(c) determining the reliability of the mutation that is registered in the mutation database by determining the mutant allele frequency of the mutation that is registered in the mutation database; and
(d) generating a result as to whether the tissue sample target nucleic acid sequence data contains a mutation, thereby detecting the mutation.

9. A computing system comprising:
one or more processors;
memory; and
one more programs, wherein the one or more programs are stored in the memory and are configured to be executed by the one or more processors for detecting a mutation in a tissue sample target nucleic acid sequence, wherein the one or more programs include instructions for detecting a mutation in a tissue sample target nucleic acid sequence comprising:
(a) obtaining a tissue sample target nucleic acid sequence data and database target nucleic acid sequence data, wherein the database target nucleic acid sequence data is located in a mutation database;
(b) comparing the tissue sample target nucleic acid sequence data with the database target nucleic acid sequence data to determine if the sample target nucleic acid sequence data contains a registered mutation from the mutation database;
(c) determining the reliability of the mutation that is registered in the mutation database by determining the mutant allele frequency of the mutation that is registered in the mutation database; and
(d) generating a result as to whether the tissue sample target nucleic acid sequence data contains a mutation, thereby detecting the mutation.

10. A method for determining whether or not a nucleic acid from a preserved tissue sample has a mutation, the method comprising the steps of:
(a) incubating the preserved tissue sample with a tissue digestion solution to form a tissue digestion mixture, wherein the tissue digestion solution is selected from the group consisting of:
(i) a tissue digestion solution comprising NaCl at a concentration of 10 mM to 140 mM, Na₂HPO₄ at a concentration of 0.5 mM to 10 mM, KH₂PO₄ at a concentration of 0.1 mM to 5 mM, and Tween 20;
(ii) a tissue digestion solution comprising NaCl at a concentration of 10 mM to 140 mM, Na₂HPO₄ at a concentration of 0.5 mM to 10 mM, KH₂PO₄ at a concentration of 0.1 mM to 5 mM, and Triton-X100;
(iii) a tissue digestion solution comprising NaCl at a concentration of 10 mM to 140 mM, Na₂HPO₄ at a concentration of 0.5 mM to 10 mM, and KH₂PO₄ at a concentration of 0.1 mM to 5 mM;
(iv) a tissue digestion solution comprising TAPS sodium salt at a concentration of 0.5 mM to 25 mM, DTT at a concentration of 0.05 mM to 5 mM, and KCl at a concentration of 0.2 mM to 200 mM;
(v) a tissue digestion solution comprising HEPES buffer at a concentration of 1 mM to 100 mM;
(vi) a tissue digestion solution comprising HEPES buffer at a concentration of 1 mM to 100 mM and Triton-X100;
(vii) a tissue digestion solution comprising HEPES buffer at a concentration of 1 mM to 100 mM and Tween 20;
(viii) a tissue digestion solution comprising TAPS sodium salt at a concentration of 0.5 mM to 25 mM, DTT at a concentration of 0.05 mM to 5 mM, KCl at a concentration of 0.2 mM to 200 mM, and Triton-X100;
(ix) a tissue digestion solution comprising a TAPS sodium salt at a concentration of 0.5 mM to 25 mM, DTT at a concentration of 0.05 mM to 5 mM, KCl at a concentration of 0.2 mM to 200 mM, and Tween 20; and
(x) a tissue digestion solution comprising a TAPS sodium salt at a concentration of 0.5 mM to 25 mM, KCl at a concentration of 0.2 mM to 200 mM, β-Mercaptoethanol at a concentration of 0.1 mM to 1 mM, and Triton-X100,
(b) heating the tissue digestion mixture at 80 to 110° C. for 1-30 minutes;
(c) adding a proteinase solution comprising a proteinase to the tissue digestion mixture to form a protein degradation mixture and incubating the protein degradation mixture at 50 to 70° C. for 1-30 minutes;
(d) incubating the protein degradation mixture at 80 to 110° C. for 1-30 minutes; thereby extracting nucleic acid from the preserved tissue sample;
(e) amplifying nucleic acid extracted from the tissue sample, the step of amplication using 5′ phosphorylated oligonucleotides that target a nucleic acid of interest;
(f) directly ligating an oligonuclotide comprising an adaptor nucleic acid and a bar code nucleic acid to each of the amplified target nucleic acids, thereby making a targeted nucleic acid amplicon library comprising tissue sample target nucleic acid;
(g) sequencing the library;
(h) obtaining a tissue sample target nucleic acid sequence data and database target nucleic acid sequence data, wherein the database target nucleic acid sequence data is located in a mutation database;
(i) comparing the tissue sample target nucleic acid sequence data with the database target nucleic acid sequence data to determine if the sample target nucleic acid sequence data contains a registered mutation from the mutation database;
(j) determining the reliability of the mutation that is registered in the mutation database by determining the mutant allele frequency of the mutation that is registered in the mutation database; and
(k) generating a result as to whether the tissue sample target nucleic acid sequence data contains a mutation, thereby detecting the mutation.
